SEQUENCE LISTING (46 sequences)

SEQ ID NO: 1 - DNA, 1161 bp, Thermotoga maritima
atggacatcctgaggaaagtggcagagattcatgggacacccacctacgtatacttcgaggaaacactgcgaaaaaggtcacgtcttgtaaaagaggtcttcgagggagtgaatctccttccaacgtttgccgtgaaagcgaacaacaatcctgttcttttgaagattctaagagaagagggtttcggcatggacgtggtgacaaagggggaactcctcgcggctaaactggcgggagttccttcccataccgttgtatggaacggcaacggaaagagcagggatcaaatggaacactttttgagagaagatgtgagaatcgtcaacgtggattcgttcgaggagatggagatctggagagaattgaacccggaaggcgtggagtatttcatcagggtgaatccggaggtcgatgcgaagacacaccctcacatctccaccggcttgaaaaagcacaagttcggaataccactggaagatctggattcgttcatggaaagattcagatcaatgaacataagaggtctccatgttcacataggatcgcagataacccgggttgaaccctttgtggaagccttcagtaaagttgttcgggcttctgaaaggtatggattcgaagagatcaacatcggcggcggctggggaataaactacagcggagaggaactcgacctgtccagttacagagaaaaggttgttcctgatttgaagagattcaaaagagtcatcgtcgaaataggaaggtacatcgtagcaccttctgggtatctgctcctcagagtggtgctcgtcaaaagaagacataacaaggcgttcgttgtagtcgatggtgggatgaatgtcctcataagaccggcactttattccgcatatcacaggatctttgtgctcggaaaacagggtaaagagatgagggcagatgtggttggtccactgtgcgaaagcggtgacgtgatcgcgtacgaccgggaacttccagaggtcgaaccgggtgacatcatcgctgtggaaaacgcgggagcttacggttacactatgtcgaacaactacaactcgaccacacgtccagctgaagtgctcgtcagagaaaacggaagaatttctctgataagaagaagggaaacggagatggatattttcaaagacgtggtgatgtga

SEQ ID NO: 2 - protein, 386 aa, Thermotoga maritima
MDILRKVAEIHGTPTYVYFEETLRKRSRLVKEVFEGVNLLPTFAVKANNNPVLLKILREEGFGMDVVTKGELLAAKLAGVPSHTVVWNGNGKSRDQMEHFLREDVRIVNVDSFEEMEIWRELNPEGVEYFIRVNPEVDAKTHPHISTGLKKHKFGIPLEDLDSFMERFRSMNIRGLHVHIGSQITRVEPFVEAFSKVVRASERYGFEEINIGGGWGINYSGEELDLSSYREKVVPDLKRFKRVIVEIGRYIVAPSGYLLLRVVLVKRRHNKAFVVVDGGMNVLIRPALYSAYHRIFVLGKQGKEMRADVVGPLCESGDVIAYDRELPEVEPGDIIAVENAGAYGYTMSNNYNSTTRPAEVLVRENGRISLIRRRETEMDIFKDVVM

SEQ ID NO: 3 - DNA, 1161 bp, Artificial sequence
  codon optimized T. maritima diaminopimelate decarboxylase gene
atggacatcctgagaaaggtcgcggagattcacggtactccgacgtacgtctacttcgaagagactttgcgtaaacgcagccgcttggtgaaagaggtctttgagggcgttaatctgctgccgacgttcgcggtgaaggcgaataacaatccggtcctgctgaagatcctgcgcgaggagggttttggtatggacgtggtcaccaagggcgagctgctggcggcaaaactggcgggtgtcccgagccataccgtcgtttggaatggtaatggcaaatcgcgtgaccagatggagcattttctgcgtgaggacgttcgtatcgttaatgtggactcttttgaagagatggaaatctggcgtgaactgaatccggagggtgtcgagtatttcatccgtgtcaacccagaagtggacgctaaaacgcatccgcacatcagcacgggcctgaagaaacacaagttcggtatcccgctggaagatctggacagcttcatggaacgtttccgtagcatgaacattcgcggcctgcacgttcacatcggttcccagattacccgcgtcgaaccgttcgttgaggcttttagcaaggtggttcgtgcgagcgagcgttatggtttcgaagaaatcaacatcggtggtggttggggcattaactactccggtgaagagctggatctgagctcttatcgtgaaaaggtggtcccggacctgaaacgcttcaagcgtgtgattgttgagattggccgctacatcgtggcgccgtctggttacttgctgctgcgtgttgtgctggtgaaacgtcgccataacaaagcctttgttgtggtggatggtggcatgaatgtgttgattcgtccggcactgtacagcgcctaccaccgcattttcgtcttgggcaagcaaggcaaagagatgcgtgcggacgtcgtcggccctctgtgcgagagcggtgatgttattgcttacgatcgtgagctgcctgaagttgaaccgggtgacatcattgccgttgagaacgcaggcgcgtacggttataccatgagcaataactataacagcaccacccgtccagccgaggtgctggttcgcgagaacggtcgtattagcctgattcgtcgccgtgaaacggaaatggatatctttaaggatgtggttatgtaa

SEQ ID NO: 4 - DNA, 1338 bp, Corynebacterium glutamicum
atggctacagttgaaaatttcaatgaacttcccgcacacgtatggccacgcaatgccgtgcgccaagaagacggcgttgtcaccgtcgctggtgtgcctctgcctgacctcgctgaagaatacggaaccccactgttcgtagtcgacgaggacgatttccgttcccgctgtcgcgacatggctaccgcattcggtggaccaggcaatgtgcactacgcatctaaagcgttcctgaccaagaccattgcacgttgggttgatgaagaggggctggcactggacattgcatccatcaacgaactgggcattgccctggccgctggtttccccgccagccgtatcaccgcgcacggcaacaacaaaggcgtagagttcctgcgcgcgttggttcaaaacggtgtgggacacgtggtgctggactccgcacaggaactagaactgttggattacgttgccgctggtgaaggcaagattcaggacgtgttgatccgcgtaaagccaggcatcgaagcacacacccacgagttcatcgccactagccacgaagaccagaagttcggattctccctggcatccggttccgcattcgaagcagcaaaagccgccaacaacgcagaaaacctgaacctggttggcctgcactgccacgttggttcccaggtgttcgacgccgaaggcttcaagctggcagcagaacgcgtgttgggcctgtactcacagatccacagcgaactgggcgttgcccttcctgaactggatctcggtggcggatacggcattgcctataccgcagctgaagaaccactcaacgtcgcagaagttgcctccgacctgctcaccgcagtcggaaaaatggcagcggaactaggcatcgacgcaccaaccgtgcttgttgagcccggccgcgctatcgcaggcccctccaccgtgaccatctacgaagtcggcaccaccaaagacgtccacgtagacgacgacaaaacccgccgttacatcgccgtggacggaggcatgtccgacaacatccgcccagcactctacggctccgaatacgacgcccgcgtagtatcccgcttcgccgaaggagacccagtaagcacccgcatcgtgggctcccactgcgaatccggcgatatcctgatcaacgatgaaatctacccatctgacatcaccagcggcgacttccttgcactcgcagccaccggcgcatactgctacgccatgagctcccgctacaacgccttcacacggcccgccgtcgtgtccgtccgcgctggcagctcccgcctcatgctgcgccgcgaaacgctcgacgacatcctctcactagaggcataa

SEQ ID NO: 5 - protein, 445 aa, Corynebacterium glutamicum
MATVENFNELPAHVWPRNAVRQEDGVVTVAGVPLPDLAEEYGTPLFVVDEDDFRSRCRDMATAFGGPGNVHYASKAFLTKTIARWVDEEGLALDIASINELGIALAAGFPASRITAHGNNKGVEFLRALVQNGVGHVVLDSAQELELLDYVAAGEGKIQDVLIRVKPGIEAHTHEFIATSHEDQKFGFSLASGSAFEAAKAANNAENLNLVGLHCHVGSQVFDAEGFKLAAERVLGLYSQIHSELGVALPELDLGGGYGIAYTAAEEPLNVAEVASDLLTAVGKMAAELGIDAPTVLVEPGRAIAGPSTVTIYEVGTTKDVHVDDDKTRRYIAVDGGMSDNIRPALYGSEYDARVVSRFAEGDPVSTRIVGSHCESGDILINDEIYPSDITSGDFLALAATGAYCYAMSSRYNAFTRPAVVSVRAGSSRLMLRRETLDDILSLEA

SEQ ID NO: 6 - DNA, 1338 bp, Artificial sequence
  codon optimized C. glutamicum diaminopimelate decarboxylase gene
atggccacggtcgaaaattttaatgagctgccggcgcacgtctggcctcgtaacgcggtccgccaagaggacggtgtggttaccgtcgccggtgttccgctgccggacctggcagaagaatatggtactccgctgttcgtggttgacgaggatgactttcgtagccgttgtcgtgatatggcgaccgcttttggtggccctggtaacgtgcattatgcctccaaggcgtttctgaccaaaacgattgcacgttgggtcgatgaagagggcctggcactggacattgcatcgattaacgaactgggcattgctctggcagcgggttttccggcgagccgtattaccgcccatggcaacaacaaaggcgtggaattcttgcgtgcgctggtgcagaatggtgttggccacgttgttctggacagcgcgcaggagctggagctgctggactacgtcgcggcaggcgaaggtaagatccaagacgtgctgatccgcgtcaaaccgggtatcgaagcgcatacgcacgaattcatcgcgaccagccacgaggatcagaaattcggtttcagcctggcctcgggtagcgcatttgaggcggcgaaagcggcaaacaatgcggagaatctgaatttggttggtctgcattgccatgtcggtagccaggttttcgacgccgagggcttcaaactggcggcagagcgtgttctgggtttgtacagccaaattcactctgagctgggcgtggcgttgccggaactggatctgggtggtggctatggcatcgcatatactgcggccgaagagccgctgaatgttgccgaggtcgcaagcgacctgctgacggcagtgggcaagatggcggctgaactgggtattgatgcgccgaccgtcctggtggaaccgggtcgtgccatcgccggtccatccacggttaccatctatgaggtgggtaccaccaaggatgttcacgtcgacgatgataagactcgccgctacattgcagtggatggtggcatgagcgacaacatccgtccagcgctgtatggtagcgagtacgatgcgcgtgttgtgagccgctttgctgaaggcgacccggtcagcacgcgtattgtcggcagccactgtgagagcggcgacatcctgattaacgatgagatttacccgtccgacatcacgagcggtgattttctggctctggccgccactggcgcgtactgctacgcgatgagcagccgctacaatgcgttcacgcgtcctgctgttgtgtctgtccgtgcgggtagcagccgcctgatgctgcgccgtgaaaccttggatgacatcttgtccctggaggcataa

SEQ ID NO: 7 - DNA, 1326 bp, Bacillus subtilis
atgacattgttcttacacggcacaagcagacaaaatcaacatggtcatttagaaatcggaggtgtggatgctctctatttagcggagaaatatggtacacctctttacgtatatgatgtggctttaatacgtgagcgtgctaaaagctttaagcaggcgtttatttctgcagggctgaaagcacaggtggcatatgcgagcaaagcattctcatcagtcgcaatgattcagctcgctgaggaagagggactttctttagatgtcgtatccggaggagagctatatacggctgttgcagcaggctttccggcagaacgcatccactttcatggaaacaataagagcagggaagaactgcggatggcgcttgagcaccgcatcggctgcattgtggtggataatttctatgaaatcgcgcttcttgaagacctatgtaaagaaacgggtcactccatcgatgttcttcttcggatcacgcccggagtagaagcgcatacgcatgactacattacaacgggccaggaagattcaaagtttggtttcgatcttcataacggacaaactgaacgggccattgaacaagtattacaatcggaacacattcagctgctgggtgtccattgccatatcggctcgcaaatctttgatacggccggttttgtgttagcagcggaaaaaatcttcaaaaaactagacgaatggagagattcatattcatttgtatccaaggtgctgaatcttggaggaggtttcggcattcgttatacggaagatgatgaaccgcttcatgccactgaatacgttgaaaaaattatcgaagctgtgaaagaaaatgcttcccgttacggttttgacattccggaaatttggatcgaaccgggccgttctctcgtgggagacgcaggcacaactctttatacggttggctctcaaaaagaagtgccgggtgtccgccaatatgtggctgtagacggaggcatgaacgacaatattcgtcctgcgctttaccaagctaaatatgaagctgcggcagccaacaggatcggagaagcgcatgacaaaacggtatcaattgccggaaagtgctgtgaaagcggagatatgctgatttgggatattgacctgccggaagtaaaagaaggcgatcttcttgccgttttttgtacaggcgcttatggatacagcatggccaacaattataaccgtattccgagacccgccgttgtatttgtcgaaaacggtgaggctcatttagtcgtgaagcgagaaacatacgaagatattgtaaaacttgatctgccatttaaaacgggtgtaaagcaataa

SEQ ID NO: 8 - protein, 441 aa, Bacillus subtilis
MTLFLHGTSRQNQHGHLEIGGVDALYLAEKYGTPLYVYDVALIRERAKSFKQAFISAGLKAQVAYASKAFSSVAMIQLAEEEGLSLDVVSGGELYTAVAAGFPAERIHFHGNNKSREELRMALEHRIGCIVVDNFYEIALLEDLCKETGHSIDVLLRITPGVEAHTHDYITTGQEDSKFGFDLHNGQTERAIEQVLQSEHIQLLGVHCHIGSQIFDTAGFVLAAEKIFKKLDEWRDSYSFVSKVLNLGGGFGIRYTEDDEPLHATEYVEKIIEAVKENASRYGFDIPEIWIEPGRSLVGDAGTTLYTVGSQKEVPGVRQYVAVDGGMNDNIRPALYQAKYEAAAANRIGEAHDKTVSIAGKCCESGDMLIWDIDLPEVKEGDLLAVFCTGAYGYSMANNYNRIPRPAVVFVENGEAHLVVKRETYEDIVKLDLPFKTGVKQ

SEQ ID NO: 9 - DNA, 1326 bp, Artificial sequence
  codon optimized B. subtilis diaminopimelate decarboxylase gene
atgaccttattcctgcacggtacctctcgccagaaccaacacggccacttggaaatcggtggtgttgacgcactgtatctggcggagaagtacggtaccccgttgtatgtctacgacgtggccctgatccgtgagcgcgcaaagagcttcaaacaggctttcattagcgctggtctgaaggcgcaagttgcgtatgcgagcaaagcgtttagcagcgttgccatgatccaactggcggaagaagagggtctgagcctggacgtcgtgtctggcggtgagctgtacaccgcggttgctgcgggcttccctgcagaacgcattcacttccatggcaacaataagagccgtgaagagctgcgtatggcgctggagcatcgtattggttgcatcgttgtggataacttttacgagattgcactgctggaagatctgtgcaaagaaacgggtcacagcatcgatgtgctgctgcgcattactccgggcgtcgaggcccacacccacgactacattacgacgggccaggaagatagcaagttcggtttcgacctgcataatggtcaaacggagcgtgccatcgaacaggtgctgcaatcggagcatattcaactgttgggtgtgcactgtcacatcggcagccagattttcgacaccgcaggctttgtcctggctgcagagaagattttcaagaaactggatgaatggcgcgattcctacagctttgtgtccaaggtgctgaatctgggcggtggttttggcatccgctataccgaagatgacgaaccgctgcacgcaacggagtacgttgagaaaatcattgaggcggtgaaagagaacgcgagccgctatggtttcgatattccggagatttggatcgagccaggccgcagcctggtgggtgacgccggcacgacgctgtatactgtcggttctcagaaagaagttccaggcgtccgtcagtatgttgctgtggacggtggtatgaacgacaatatccgtccggcgctgtatcaggcgaaatacgaggcagctgcagcgaaccgtatcggcgaagcccacgacaaaaccgtcagcatcgcgggcaaatgctgtgaaagcggtgatatgctgatttgggacatcgatttgccggaggtcaaagagggcgacttgctggctgttttctgtaccggtgcgtatggttacagcatggccaataactacaatcgtattccgcgtccggccgttgtgtttgttgagaatggtgaagcacatttggttgtgaagcgtgaaacctacgaggacatcgtcaaactggatctgccgtttaagaccggtgtcaagcaataa

SEQ ID NO: 10 - DNA, 1248 bp, Pseudomonas putida
atgaacgctttcaactaccgcgacggccagctgttcgcggaaggggtggccctgtcggccgtcgccgaacgtttcggcacccccacctacgtgtattcgcgcgcccacatcgaggcccagtaccgcagctacaccgacgccctgcaaggcgccgagcacctggtgtgcttcgcggtcaaggccaactccaacctcggcgtgctgaacgtgctggcacgcctgggcgcaggcttcgacattgtctccggcggtgagctggagcgcgtgctggctgctggcgggcgcgccgaccgcgtggtgttctccggcgtcggcaaaacccgcgaagacatgcgccgcgccctggaagtgggcgtgcactgcttcaacgtcgaatccaccgacgagctggagcgcctgcaggtcgtggccgccgaaatgggcaaggtcgccccggtgtcgctgcgggtaaacccggatgtagacgccggcacccacccgtacatctccacgggccttaaagaaaacaagttcggtatcgccatcgccgacgccgaggccatctacgtgcgtgccgcgcagcttccgaacctggaagtggtcggcgtcgactgccacatcggctcacagctgaccaccgtggagccgttcctcgatgccctcgaccgcctgctggacctggtcgatcgcctcgccgactgcggcatccacctgcgccatctggacctgggtggcggcgttggcgtgcgctaccgcgacgaggagccaccgctggtggccgactacatcaaggctattcgcgaacgcgtaggcaagcgcgacctggccctggtgttcgagccgggccgctacatcgtggccaacgccggcgtgttgctgacccgcgtggaatacctcaagcacaccgaacacaaagacttcgccatcatcgatgcggcaatgaacgacctgatccgcccggccctttaccaggcctggatgggtgtcagcgcggtcatcccacgcgaaggcgaagggcgtgcctacgacctggtcggcccaatctgcgagaccggcgacttcctcggcaaggaccgcgtgttgaacctggccgaaggcgacctgctggccgtgcagtccgcgggcgcctatggttttgtcatgagttccaactacaacacccgtggccgttgcgctgaaatcctggtcgacggcgaccaggcgttcgaagtacgccgccgcgagaccatcgccgaactgtacgctggcgaaagcctgctgccggagtaa

SEQ ID NO: 11 - protein, 415 aa, Pseudomonas putida
MNAFNYRDGQLFAEGVALSAVAERFGTPTYVYSRAHIEAQYRSYTDALQGAEHLVCFAVKANSNLGVLNVLARLGAGFDIVSGGELERVLAAGGRADRVVFSGVGKTREDMRRALEVGVHCFNVESTDELERLQVVAAEMGKVAPVSLRVNPDVDAGTHPYISTGLKENKFGIAIADAEAIYVRAAQLPNLEVVGVDCHIGSQLTTVEPFLDALDRLLDLVDRLADCGIHLRHLDLGGGVGVRYRDEEPPLVADYIKAIRERVGKRDLALVFEPGRYIVANAGVLLTRVEYLKHTEHKDFAIIDAAMNDLIRPALYQAWMGVSAVIPREGEGRAYDLVGPICETGDFLGKDRVLNLAEGDLLAVQSAGAYGFVMSSNYNTRGRCAEILVDGDQAFEVRRRETIAELYAGESLLPE

SEQ ID NO: 12 - DNA, 28 bp, Artificial sequence
  Forward primer lysA P. putida
gccatatgaacgctttcaactaccgcga

SEQ ID NO: 13 - DNA, 29 bp, Artificial sequence
  Reverse primer lysA P. putida
gcaagcttactccggcagcaggctttcgc

SEQ ID NO: 14 - DNA, 1362 bp, Vibrio fluvialis
atgaacaaaccgcaaagctgggaagcccgggccgagacctattcgctctatggtttcaccgacatgccttcgctgcatcagcgcggcacggtcgtcgtgacccatggcgagggaccctatatcgtcgatgtgaatggccggcgttatctggacgccaactcgggcctgtggaacatggtcgcgggctttgaccacaaggggctgatcgacgccgccaaggcccaatacgagcgttttcccggttatcacgcctttttcggccgcatgtccgatcagacggtaatgctgtcggaaaagctggtcgaggtgtcgccctttgattcgggccgggtgttctatacaaactcggggtccgaggcgaatgacaccatggtcaagatgctatggttcctgcatgcagccgagggcaaaccgcaaaagcgcaagatcctgacccgctggaacgcctatcacggcgtgaccgccgtttcggccagcatgaccggcaagccctataattcggtctttggcctgccgctgccgggctttgtgcatctgacctgcccgcattactggcgctatggcgaagagggcgaaaccgaagagcagttcgtcgcccgcctcgcccgcgagctggaggaaacgatccagcgcgagggcgccgacaccatcgccggtttctttgccgaaccggtgatgggcgcgggcggcgtgattcccccggccaagggctatttccaggcgatcctgccaatcctgcgcaaatatgacatcccggtcatctcggacgaggtgatctgcggtttcggacgcaccggtaacacctggggctgcgtgacctatgactttacacccgatgcaatcatctcgtccaagaatcttacagcgggctttttccccatgggggcggtgatccttggcccggaactttccaaacggctggaaaccgcaatcgaggcgatcgaggaattcccccatggctttaccgcctcgggccatccggtcggctgtgctattgcgctgaaagcaatcgacgtggtgatgaatgaagggctggctgagaacgtccgccgccttgccccccgtttcgaggaaaggctgaaacatatcgccgagcgcccgaacatcggtgaatatcgcggcatcggcttcatgtgggcgctggaggctgtcaaggacaaggcaagcaagacgccgttcgacggcaacctgtcggtcagcgagcgtatcgccaatacctgcaccgatctggggctgatttgccggccgcttggtcagtccgtcgtcctttgtccgccctttatcctgaccgaggcgcagatggatgagatgttcgataaactcgaaaaagcccttgataaggtctttgccgaggttgcctga

SEQ ID NO: 15 - protein, 453 aa, Vibrio fluvialis
MNKPQSWEARAETYSLYGFTDMPSLHQRGTVVVTHGEGPYIVDVNGRRYLDANSGLWNMVAGFDHKGLIDAAKAQYERFPGYHAFFGRMSDQTVMLSEKLVEVSPFDSGRVFYTNSGSEANDTMVKMLWFLHAAEGKPQKRKILTRWNAYHGVTAVSASMTGKPYNSVFGLPLPGFVHLTCPHYWRYGEEGETEEQFVARLARELEETIQREGADTIAGFFAEPVMGAGGVIPPAKGYFQAILPILRKYDIPVISDEVICGFGRTGNTWGCVTYDFTPDAIISSKNLTAGFFPMGAVILGPELSKRLETAIEAIEEFPHGFTASGHPVGCAIALKAIDVVMNEGLAENVRRLAPRFEERLKHIAERPNIGEYRGIGFMWALEAVKDKASKTPFDGNLSVSERIANTCTDLGLICRPLGQSVVLCPPFILTEAQMDEMFDKLEKALDKVFAEVA

SEQ ID NO: 16 - DNA, 1362 bp, Vibrio fluvialis
atgaataaaccacagtcttgggaagctcgtgctgaaacctatagcctgtacggctttaccgatatgccgtctctgcaccagcgtggtactgtagtggtaacgcacggtgagggcccgtacatcgtggacgttaatggccgccgttacctggatgcaaacagcggcctgtggaacatggttgcgggcttcgaccacaaaggcctgatcgatgccgcaaaagcgcagtacgaacgcttcccgggttatcacgcgttctttggccgtatgagcgaccagactgtgatgctgagcgaaaaactggttgaagtgtccccgttcgatagcggtcgtgtcttttacactaactctggcagcgaggctaacgataccatggttaagatgctgtggttcctgcacgcagcggaaggcaaacctcagaaacgtaaaattctgacccgttggaacgcttatcacggtgtgactgctgtttccgcatctatgaccggtaaaccgtataacagcgtgttcggtctgccgctgcctggcttcgtgcatctgacctgcccgcactactggcgttatggtgaggaaggcgaaactgaggaacagttcgtggcgcgtctggctcgtgaactggaagaaaccattcaacgcgaaggtgcagatactatcgcgggcttctttgcggagcctgttatgggtgccggcggtgtgattccgccggcgaagggctatttccaggcaatcctgccgatcctgcgcaagtacgacattccggttatttctgacgaagtgatctgcggcttcggccgcaccggtaacacctggggctgcgtgacgtatgacttcactccggacgcaatcattagctctaaaaacctgactgcgggtttcttccctatgggcgccgtaatcctgggcccagaactgtctaagcgcctggaaaccgccatcgaggcaatcgaagagttcccgcacggtttcactgctagcggccatccggtaggctgcgcaatcgcgctgaaggcgatcgatgttgtcatgaacgagggcctggcggaaaacgtgcgccgcctggcgccgcgttttgaagaacgtctgaaacacattgctgagcgcccgaacattggcgaatatcgcggcatcggtttcatgtgggccctggaagcagttaaagataaagctagcaagaccccgttcgacggcaacctgtccgtgagcgaacgtatcgctaatacctgtacggacctgggtctgatctgccgtccgctgggtcagtccgtagttctgtgcccaccatttatcctgaccgaagcgcagatggatgaaatgttcgataaactggagaaagctctggataaagtgttcgctgaagtcgcgtaa

SEQ ID NO: 17 - DNA, 1350 bp, Bacillus weihenstephanensis
gtgcaagcgacggagcaaacacaaagtttgaaaaaaacagatgaaaagtacctttggcatgcgatgagaggagcagcccctagtccaacgaatttaattatcacaaaagcagaaggggcatgggtgacggatattgatggaaaccgttatttagacggtatgtccggtctttggtgcgtgaatgttgggtatggtcgaaaagaacttgcaagagcggcgtttgaacagcttgaagaaatgccgtatttccctctgactcaaagtcatgttcctgctattaaattagcagaaaaattgaatgaatggcttgatgatgaatacgtcattttcttttctaacagtggatcggaagcgaatgaaacagcatttaaaattgctcgtcaatatcatcaacaaaaaggtgatcatggacgctataagtttatttcccgctaccgcgcttatcacggtaactcaatgggagctcttgcagcaacaggtcaagcacagcgaaagtataaatatgaaccactcgggcaaggattcctgcatgtagcaccgcctgatacgtatcgaaatccagaggatgttcatacactggcaagtgctgaggaaatcgatcgtgtcatgacatgggagttaagccaaacagtagccggtgtgattatggagccaatcattactgggggcggaattttaatgcctcctgatggatatatgggaaaagtaaaagaaatttgcgagaagcacggtgcgttgctcatttgtgatgaagttatatgtggatttggccggacagggaagccatttggatttatgaattatggcgtcaaaccagatatcattacaatggcaaaaggtattacaagtgcgtatcttcctttgtcagcaacagcagttagacgagaggtttatgaggcattcgtaggtagtgatgattatgatcgcttccgccatgtaaatacgttcggagggaatcctgctgcttgcgctttagctttgaagaatttagaaattatggagaatgagaaactcattgaacgttccaaagaattgggtgaacgactgttatatgagctagaggatgtaaaagagcatccaaacgtaggggatgttcgcggaaagggccttcttttaggcattgaactagtggaagataagcaaacaaaagaaccggcttccattgaaaagatgaacaaagtcatcaatgcttgtaaagaaaaaggtctaattattggtaaaaatggtgacactgtcgcaggttacaataatattttgcagcttgcacctccattaagcatcacagaggaagactttacttttatcgttaaaacaatgaaagaatgtttatcccgcattaacgggcagtaa

SEQ ID NO: 18 - protein, 449 aa, Bacillus weihenstephanensis
VQATEQTQSLKKTDEKYLWHAMRGAAPSPTNLIITKAEGAWVTDIDGNRYLDGMSGLWCVNVGYGRKELARAAFEQLEEMPYFPLTQSHVPAIKLAEKLNEWLDDEYVIFFSNSGSEANETAFKIARQYHQQKGDHGRYKFISRYRAYHGNSMGALAATGQAQRKYKYEPLGQGFLHVAPPDTYRNPEDVHTLASAEEIDRVMTWELSQTVAGVIMEPIITGGGILMPPDGYMGKVKEICEKHGALLICDEVICGFGRTGKPFGFMNYGVKPDIITMAKGITSAYLPLSATAVRREVYEAFVGSDDYDRFRHVNTFGGNPAACALALKNLEIMENEKLIERSKELGERLLYELEDVKEHPNVGDVRGKGLLLGIELVEDKQTKEPASIEKMNKVINACKEKGLIIGKNGDTVAGYNNILQLAPPLSITEEDFTFIVKTMKECLSRINGQ

SEQ ID NO: 19 - DNA, 1350 bp, Bacillus weihenstephanensis
atgcaggctaccgaacaaacccaatctctgaaaaagactgacgaaaaatatctgtggcacgcgatgcgcggtgcagctccgtctccgaccaacctgattattaccaaagctgaaggcgcgtgggtgaccgacattgacggtaaccgttatctggatggcatgagcggcctgtggtgtgttaatgtcggttatggccgtaaggagctggcgcgcgcggcatttgaacaactggaagaaatgccgtacttcccgctgactcaaagccatgtgccggctatcaaactggcggaaaaactgaacgaatggctggacgacgaatacgtgattttcttctctaattctggctccgaagcaaacgaaaccgcattcaaaatcgcccgtcaatatcaccagcagaaaggtgaccacggccgctataaattcatcagccgttatcgtgcataccatggtaattctatgggtgcgctggctgctaccggtcaggctcagcgcaaatacaagtacgaaccgctgggtcagggttttctgcacgttgcaccaccggatacctaccgtaacccggaagacgtccacaccctggcttctgccgaagaaatcgatcgtgttatgacctgggagctgtcccagactgttgcgggtgttatcatggaacctattattaccggtggtggcattctgatgccgccggacggttatatgggtaaagtcaaggaaatctgcgaaaaacacggcgcgctgctgatctgcgatgaagttatctgtggcttcggtcgcaccggcaaaccatttggcttcatgaattatggcgtaaaacctgacattattaccatggctaaaggcattacttccgcttatctgccgctgagcgcgaccgcagttcgccgcgaagtttatgaagcgtttgttggttctgatgattacgaccgtttccgtcatgtaaacacgtttggcggtaacccagcggcatgtgcgctggcgctgaaaaacctggaaatcatggaaaacgaaaagctgatcgaacgtagcaaagaactgggtgaacgtctgctgtacgaactggaagatgtcaaagaacacccgaacgtgggcgatgttcgcggtaaaggcctgctgctgggtattgaactggttgaagacaaacagaccaaggaaccggcttccattgaaaagatgaacaaagtgattaacgcgtgcaaagagaaaggcctgatcattggtaagaacggtgataccgtggcaggttataacaacattctgcagctggcgccgcctctgagcatcactgaagaagatttcaccttcatcgtcaaaactatgaaggagtgcctgagccgcatcaatggtcagtaa

SEQ ID NO: 20 - DNA, 1371 bp, Pseudomonas aeruginosa
atgaacagccaaatcaccaacgccaagacccgtgagtggcaggcgttgagccgcgaccaccatctgccgccgttcaccgactacaagcagttgaacgagaagggcgcgcggatcatcaccaaggccgaaggcgtctatatctgggacagcgagggcaacaagatcctcgatgcgatggccggcctctggtgcgtcaacgtcggctacggccgcgaggagctggtccaggccgccacccggcagatgcgcgagttgccgttctacaacctgttcttccagaccgcccacccgccggtggtcgagctggccaaggcgatcgccgacgtcgctccggaaggcatgaaccacgtgttcttcaccggctccggctccgaggccaacgacaccgtgctgcgtatggtccgccactattgggcgaccaagggccagccgcagaagaaagtggtgatcggccgctggaacggctaccacggctccaccgtcgccggcgtcagcctgggcggcatgaaggcgttgcatgagcagggtgatttccccatcccgggcatcgtccacatcgcccagccctactggtacggcgagggcggcgacatgtcgccggacgagttcggcgtctgggccgccgagcagttggagaagaagattctcgaagtgggcgaggaaaacgtcgccgccttcatcgccgagccgatccagggcgccggcggcgtgatcgtcccgccggacacctactggccgaagatccgcgagatcctcgccaagtacgacatcctgttcatcgccgacgaagtgatctgcggcttcggccgtaccggcgagtggttcggcagccagtactacggcaacgccccggacctgatgccgatcgccaagggcctcacctccggctacatccccatgggcggggtggtggtgcgcgacgagatcgtcgaagtgctcaaccagggcggcgagttctaccacggcttcacctattccggtcacccggtggcggccgccgtggccctggagaacatccgcatcctgcgcgaagagaagatcatcgagaaggtgaaggcggaaacggcaccgtatttgcagaaacgctggcaggagctggccgaccacccgttggtgggcgaagcgcgcggggtcggcatggtcgccgccctggagctggtcaagaacaagaagacccgcgagcgtttcaccgacaagggcgtcgggatgctgtgccgggaacattgtttccgcaacggtttgatcatgcgcgcggtgggcgacactatgattatctcgccgccgctggtgatcgatccgtcgcagatcgatgagttgatcaccctggcgcgcaagtgcctcgatcagaccgccgccgccgtcctggcttga

SEQ ID NO: 21 - protein, 456 aa, Pseudomonas aeruginosa
MNSQITNAKTREWQALSRDHHLPPFTDYKQLNEKGARIITKAEGVYIWDSEGNKILDAMAGLWCVNVGYGREELVQAATRQMRELPFYNLFFQTAHPPVVELAKAIADVAPEGMNHVFFTGSGSEANDTVLRMVRHYWATKGQPQKKVVIGRWNGYHGSTVAGVSLGGMKALHEQGDFPIPGIVHIAQPYWYGEGGDMSPDEFGVWAAEQLEKKILEVGEENVAAFIAEPIQGAGGVIVPPDTYWPKIREILAKYDILFIADEVICGFGRTGEWFGSQYYGNAPDLMPIAKGLTSGYIPMGGVVVRDEIVEVLNQGGEFYHGFTYSGHPVAAAVALENIRILREEKIIEKVKAETAPYLQKRWQELADHPLVGEARGVGMVAALELVKNKKTRERFTDKGVGMLCREHCFRNGLIMRAVGDTMIISPPLVIDPSQIDELITLARKCLDQTAAAVLA

SEQ ID NO: 22 - DNA, 1365 bp, Pseudomonas syringae
atgagtgccaacaacccgcaaaccctcgaatggcaggccctgagcagcgagcatcacctggcaccgttcagcgactacaaacaactgaaagagaaaggcccgcgcatcatcacccgtgccgagggcgtttatctgtgggacagcgagggcaacaagatcctcgatggcatgtccggcctgtggtgcgtggccatcggttatggccgcgaagaactggccgacgcagccagcaaacagatgcgcgagctgccgtactacaacctgttcttccagaccgcccacccgccggtgctggaactggccaaggccatctccgacatcgctcccgagggcatgaaccatgtgttcttcaccggttcaggctctgaaggcaatgacacgatgctgcgcatggttcgtcattactgggcgctgaaaggccagccgaacaagaaaaccatcatcagccgcgtcaatggctaccacggctccaccgtcgccggtgccagcctgggtggcatgacctacatgcacgaacagggcgacctgccgatcccgggggtggtgcacattccacagccttactggttcggcgaaggcggcgacatgacgccggacgagttcggcatctgggcggccgagcaactggaaaagaaaattctcgagctgggcgtcgagaacgtcggtgcgttcattgccgagccaatccagggcgcgggcggtgtgattgtcccgcctgattcctactggccgaagatcaaggaaatcctttcccgctacgacatcctgttcgccgccgatgaggtgatttgtggcttcgggcgtaccagtgagtggttcggtagcgatttctatggcctcaggccggacatgatgaccatcgccaaaggcctgacctccggttacgtaccgatgggcggcctgatcgtgcgcgatgaaatcgttgcggtgctcaatgagggtggcgatttcaatcacggctttacctactccgggcacccggtggcggccgcggttgcgctggagaacatccgtatcctgcgcgaagaaaagatcgtcgaacgggtcaggtcggaaacggcaccgtatttgcaaaagcgtttgcgtgagttgagcgatcatccgctggtgggcgaagtccggggtgtcgggctgctcggggccattgagctggtgaaggacaagaccacccgcgagcgctataccgacaagggcgcgggaatgatctgtcgaaccttctgcttcgacaatggcctgatcatgcgggctgtgggcgataccatgatcattgcgccgccactggtgatcagttttgcgcaaatcgatgagctggtagagaaggcgcgcacgtgtctggatctgacgctggcggtgttgcagggctga

SEQ ID NO: 23 - protein, 454 aa, Pseudomonas syringae
MSANNPQTLEWQALSSEHHLAPFSDYKQLKEKGPRIITRAEGVYLWDSEGNKILDGMSGLWCVAIGYGREELADAASKQMRELPYYNLFFQTAHPPVLELAKAISDIAPEGMNHVFFTGSGSEGNDTMLRMVRHYWALKGQPNKKTIISRVNGYHGSTVAGASLGGMTYMHEQGDLPIPGVVHIPQPYWFGEGGDMTPDEFGIWAAEQLEKKILELGVENVGAFIAEPIQGAGGVIVPPDSYWPKIKEILSRYDILFAADEVICGFGRTSEWFGSDFYGLRPDMMTIAKGLTSGYVPMGGLIVRDEIVAVLNEGGDFNHGFTYSGHPVAAAVALENIRILREEKIVERVRSETAPYLQKRLRELSDHPLVGEVRGVGLLGAIELVKDKTTRERYTDKGAGMICRTFCFDNGLIMRAVGDTMIIAPPLVISFAQIDELVEKARTCLDLTLAVLQG

SEQ ID NO: 24 - DNA, 1365 bp, Artificial sequence
  Pseudomonas syringae codon optimised aminotransferase gene
atgtctgctaacaatccacaaactctggaatggcaggcactgagctccgaacatcacctggctccgttctccgactacaaacaactgaaagagaaaggcccgcgtatcattacccgcgctgaaggtgtgtacctgtgggattctgaaggcaacaaaattctggacggtatgagcggcctgtggtgcgtagcaatcggttatggccgtgaagaactggctgacgcggcgagcaaacagatgcgtgaactgccgtattataacctgttcttccaaaccgcacacccgccggttctggaactggctaaagctatcagcgatatcgcaccggagggcatgaatcacgtcttcttcactggttccggtagcgaaggcaacgacacgatgctgcgcatggtacgtcactattgggcgctgaagggccagccgaacaagaaaacgattatcagccgtgtaaacggttatcacggcagcaccgttgcgggtgcgagcctgggcggtatgacctacatgcacgaacagggtgacctgccgatcccgggtgtagtgcacattccgcagccgtattggttcggtgaaggcggtgacatgacgccggacgaattcggcatctgggcggcagagcagctggaaaagaaaatcctggaactgggcgtggaaaacgtcggcgcgttcatcgcggaaccgattcagggcgcgggcggcgtaattgttccgccggacagctactggccaaaaatcaaagagatcctgtctcgttacgacatcctgttcgccgcagacgaagtgatctgcggttttggccgcacctctgaatggttcggctccgacttctacggtctgcgtccggacatgatgaccatcgccaaaggcctgacctccggttatgttcctatgggtggcctgatcgtgcgcgacgaaattgttgcggttctgaacgaaggcggcgatttcaaccacggcttcacctattccggtcacccagttgctgctgctgtagcactggaaaacatccgcatcctgcgtgaagaaaagatcgtagaacgcgtacgttccgaaaccgcaccttacctgcagaagcgcctgcgcgaactgagcgaccaccctctggtaggtgaagttcgcggcgtgggcctgctgggcgcgatcgagctggtgaaagacaaaactacccgtgaacgttacaccgacaaaggcgcaggcatgatctgccgtaccttttgcttcgataacggtctgatcatgcgcgcagtcggtgataccatgatcattgctccgcctctggttatttcttttgcccagattgatgagctggtcgaaaaagcgcgcacttgtctggatctgactctggctgttctgcagggttaa

SEQ ID NO: 25 - DNA, 849 bp, Bacillus subtilis
atgaaggttttagtcaatggccggctgattgggcgcagtgaagcatcaatcgatttggaagatcgcggttatcagtttggtgacggcatctatgaagtgatcagggtgtacaaaggagtattgttcggcttacgtgagcatgcagagcgttttttcagaagtgctgctgaaatcggaatttcactgccattcagtatagaagatctcgagtgggacctgcaaaagcttgtacaggaaaatgcggtcagtgagggagcggtatacattcagacaacaagaggtgtggccccgcgaaaacaccagtatgaagccggcctcgagccgcagactactgcctatacgtttacggtgaaaaaaccggagcaagagcaggcatacggagtggcggccattacagatgaggatcttcgctggttaagatgtgatatcaaaagtctgaatttactgtataatgtcatgacgaagcaaagggcctatgaagccggagcatttgaagccattttacttagggacggcgttgttacggagggtacatcctctaacgtttatgccgttatcaacggcacagtgcgaacacatccggctaatcggctcattctcaatggaattacacggatgaatattttaggactgattgagaagaatgggatcaaactggatgagactcctgtcagtgaagaagagttgaaacaggcggaagagatctttatttcgtcaacgacggcagaaattattccggtcgtgacgctcgatggacaatcgatcggaagcgggaaacccggaccggtgaccaaacagcttcaggctgcttttcaagaaagcattcaacaggctgctagcatttcataa

SEQ ID NO: 26 - protein, 282 aa, Bacillus subtilis
MKVLVNGRLIGRSEASIDLEDRGYQFGDGIYEVIRVYKGVLFGLREHAERFFRSAAEIGISLPFSIEDLEWDLQKLVQENAVSEGAVYIQTTRGVAPRKHQYEAGLEPQTTAYTFTVKKPEQEQAYGVAAITDEDLRWLRCDIKSLNLLYNVMTKQRAYEAGAFEAILLRDGVVTEGTSSNVYAVINGTVRTHPANRLILNGITRMNILGLIEKNGIKLDETPVSEEELKQAEEIFISSTTAEIIPVVTLDGQSIGSGKPGPVTKQLQAAFQESIQQAASIS

SEQ ID NO: 27 - DNA, 1344 bp, Bacillus subtilis
atgactcatgatttgatagaaaaaagtaaaaagcacctctggctgccatttacccaaatgaaagattatgatgaaaaccccttaatcatcgaaagcgggactggaatcaaagtcaaagacataaacggcaaggaatactatgacggtttttcatcggtttggcttaatgtccacggacaccgcaaaaaagaactagatgacgccataaaaaaacagctcggaaaaattgcgcactccacgttattgggcatgaccaatgttccagcaacccagcttgccgaaacattaatcgacatcagcccaaaaaagctcacgcgggtcttttattcagacagcggcgcagaggcgatggaaatagccctaaaaatggcgtttcagtattggaagaacatcgggaagcccgagaaacaaaaattcatcgcaatgaaaaacgggtatcacggtgatacgattggcgccgtcagtgtcggttcaattgagctttttcaccacgtatacggcccgttgatgttcgagagttacaaggccccgattccttatgtgtatcgttctgaaagcggtgatcctgatgagtgccgtgatcagtgcctccgagagcttgcacagctgcttgaggaacatcatgaggaaattgccgcgctttccattgaatcaatggtacaaggcgcgtccggtatgatcgtgatgccggaaggatatttggcaggcgtgcgcgagctatgtacaacatacgatgtcttaatgatcgttgatgaagtcgctacaggctttggccgtacaggaaaaatgtttgcgtgcgagcacgagaatgtccagcctgatctgatggctgccggtaaaggcattacaggaggctatttgccaattgccgttacgtttgccactgaagacatctataaggcattctatgatgattatgaaaacctaaaaacctttttccatggccattcctatacaaatcagcttggctgtgcggttgcgcttgaaaatctggcattatttgaatctgaaaacattgtggaacaagtagcggaaaaaagtaaaaagctccattttcttcttcaagatctgcacgctcttcctcatgttggggatattcggcagcttggctttatgtgcggtgcagagcttgtacgatcaaaggaaactaaagaaccttacccggctgatcggcggattggatacaaagtttccttaaaaatgagagagttaggaatgctgacaagaccgcttggggacgtgattgcatttcttcctcctcttgccagcacagctgaagagctctcggaaatggttgccattatgaaacaagcgatccacgaggttacgagccttgaagattga

SEQ ID NO: 28 - protein, 448 aa, Bacillus subtilis
MTHDLIEKSKKHLWLPFTQMKDYDENPLIIESGTGIKVKDINGKEYYDGFSSVWLNVHGHRKKELDDAIKKQLGKIAHSTLLGMTNVPATQLAETLIDISPKKLTRVFYSDSGAEAMEIALKMAFQYWKNIGKPEKQKFIAMKNGYHGDTIGAVSVGSIELFHHVYGPLMFESYKAPIPYVYRSESGDPDECRDQCLRELAQLLEEHHEEIAALSIESMVQGASGMIVMPEGYLAGVRELCTTYDVLMIVDEVATGFGRTGKMFACEHENVQPDLMAAGKGITGGYLPIAVTFATEDIYKAFYDDYENLKTFFHGHSYTGNQLGCAVALENLALFESENIVEQVAEKSKKLHFLLQDLHALPHVGDIRQLGFMCGAELVRSKETKEPYPADRRIGYKVSLKMRELGMLTRPLGDVIAFLPPLASTAEELSEMVAIMKQAIHEVTSLED

SEQ ID NO: 29 - DNA, 1464 bp, Rhodobacter sphaeroides
atgcccggttgcgggggcttgcccgggaatgaaccgaaatgcggacgagaggggaggtcggcgatgacgcggaatgacgcgacgaatgctgccggagcggtgggcgcggcgatgcgggatcacatcctcttgcctgcacaggaaatggcgaagctcggcaagtccgcgcagccggtgctgactcatgccgagggcatctatgtccataccgaggacggccgccgcctgatcgacgggccggcgggcatgtggtgcgcgcaggtgggctacggccgccgcgagatcgtcgatgccatggcgcatcaggcgatggtgctgccctatgcctcgccctggtatatggccacgagccccgcggcgcggctggcggagaagatcgccacgctgacgccgggcgatctcaaccggatctttttcaccacgggcgggtcgaccgcggtggacagcgcgctgcgcttctcggaattctacaacaacgtgctgggccggccgcagaagaagcgcatcatcgtgcgctacgacggctatcacggctcgacggcgctcaccgccgcctgcaccggccgcaccggcaactggccgaacttcgacatcgcgcaggaccggatctcgttcctctcgagccccaatccgcgccacgccggcaaccgcagccaggaggcgttcctcgacgatctggtgcaggaattcgaggaccggatcgagagcctcggccccgacacgatcgcggccttcctggccgagccgatcctcgcctcgggcggcgtcattattccgcccgcaggctatcatgcgcgcttcaaggcgatctgcgagaagcacgacatcctctatatctcggacgaggtggtgacgggcttcggccgttgcggcgagtggttcgcctcggagaaggtgttcggggtggtgccggacatcatcaccttcgccaagggcgtgacctcgggctatgtgccgctcggcggccttgcgatctccgaggcggtgctggcgcggatctcgggcgagaatgccaagggaagctggttcaccaacggctatacctacagcaatcagccggtggcctgcgccgcggcgcttgccaacatcgagctgatggagcgcgagggcatcgtcgatcaggcgcgcgagatggcggactatttcgccgcggcgctggcttcgctgcgcgatctgccgggcgtggcggaaacccggtcggtgggcctcgtgggttgcgtgcaatgcctgctcgacccgacccgggcggacggcacggccgaggacaaggccttcaccctgaagatcgacgagcgctgcttcgagctcgggctgatcgtgcgcccgctgggcgatctctgcgtgatctcgccgccgctcatcatctcgcgcgcgcagatcgacgagatggtcgcgatcatgcggcaggccatcaccgaagtgagcgccgcccacggtctgaccgcgaaagaaccggccgccgtc

SEQ ID NO: 30 - protein, 488 aa, Rhodobacter sphaeroides
MPGCGGLPGNEPKCGREGRSAMTRNDATNAAGAVGAAMRDHILLPAQEMAKLGKSAQPVLTHAEGIYVHTEDGRRLIDGPAGMWCAQVGYGRREIVDAMAHQAMVLPYASPWYMATSPAARLAEKIATLTPGDLNRIFFTTGGSTAVDSALRFSEFYNNVLGRPQKKRIIVRYDGYHGSTALTAACTGRTGNWPNFDIAQDRISFLSSPNPRHAGNRSQEAFLDDLVQEFEDRIESLGPDTIAAFLAEPILASGGVIIPPAGYHARFKAICEKHDILYISDEVVTGFGRCGEWFASEKVFGVVPDIITFAKGVTSGYVPLGGLAISEAVLARISGENAKGSWFTNGYTYSNQPVACAAALANIELMEREGIVDQAREMADYFAAALASLRDLPGVAETRSVGLVGCVQCLLDPTRADGTAEDKAFTLKIDERCFELGLIVRPLGDLCVISPPLIISRAQIDEMVAIMRQAITEVSAAHGLTAKEPAAV

SEQ ID NO: 31 - DNA, 831 bp, Legionella pneumophila
atgagtatcgcatttgttaacggcaagtattgttgtcaatctgaagcaaaaatttcaatatttgatcgagggtttctttttggtgactcggtttatgaagtgctgcctgtttaccatgggcagccttactttgtagaccaacatcttgaccgattattctcaaatatgaaaaaaattaagatgattataccaaattatgattggcatggtttaattcatagactaatatcagaaaataatggcggtaatttacaagtatatatccaagtcacacgagggaatcaaggggtgcgcaagcatgatatccctacttccatcacaccttctgttatcttcactatgcataatccatttcccaccctcgaagataaggaacagggaatgtcagcaaaactggttgaagattttcggtggatgagatgtgatataaaaactacttctttaattgccaatatattactgaatgatgaggctgtatctgcaggattccacactgcaattcttgcccggaacggtctaattgagggaagtagtaccaacgtatttattgtcgcacaggatggtgttattaagacaccacccatgaataatttctgtttaccaggaattactcggcaagttgttattgaaataattaaaaaattagatttaaagttcagagaaatagaaattagcatttcagagcttttttctgctcaggaagtttggataacaagtacgacaaaagaagtattccctattacaaagattaatgactctttgattaatggcggaaaagttggcgaatattggcggataattaatgattcctaccaacaactagtaaactaa

SEQ ID NO: 32 - protein, 278 aa, Legionella pneumophila
MSIAFVNGKYCCQSEAKISIFDRGFLFGDSVYEVLPVYHGQPYFVDQHLDRLFSNMKKIKMIIPNYDWHGLIHRLISENNGGNLQVYIQVTRGNQGVRKHDIPTSITPSVIAFTMHNPFPTLEDKEQGMSAKLVEDFRWMRCDIKTTSLIANILLNDEAVSAGFHTAILARNGLITEGSSTNVFIVAQDGVIKTPPMNNFCLPGITRQVVIEIIKKLDLKFREIEISISELFSAQEVWITSTTKEVFPITKINDSLINGGKVGEYWRIINDSYQQLVN

SEQ ID NO: 33 - DNA, 840 bp, Nitrosomonas europaea
atgatttacctcaatggcaaatttctgccgatggaacaggctaccgttccagtgctggatagaggcttcatcttcggtgatggtgtctatgaagtcataccggtttattcacgtaaaccgttccggctgggcgaacatctttcccggctgcagcacagtctggatggcatacgtctccagaatccgcacgaagaacaatgggctggtctgatcgaacgcatcatcgagctgaatggtgatgatcagtacctttacctgcacattacacgcggggtggcaaaacgtgaccatgcctttcctcgcgaagtaacgcccactgtcttcatcagcaacccgcttccggctccacctgcaaaattgctcgtttccggagtttcagcgattaccgccagggataatcgctgggggcgctgtgatatcaaagccatttcactgttgccaaatatcttattgcgccagcttgccgtggacgcacaagccatggaaacgatcctgttacgcgatggtctgttgaccggggccgccagcaatattttcatcgtaaaagacgacctgctgctgaccccccccaaagatcaccgtatattgcctggcattacttatgatgtagtactggaactggctgaaacacatggtgttccacatgcgacaagagaaatatcagagcttgagttacgtactgcacgggaaatcatgctgacttctaccaaagaaattctcccgatcacacagctggatggacaaccgatcaatggcaccccagggccagtatttcagcaactggatcggctctatgcatataagctggaagtcatgcgcgggcatgctccacgccagtaa

SEQ ID NO: 34 - protein, 286 aa, Nitrosomonas europaea
MIYLNGKFLPMEQATVPVLDRGFIFGDGVYEVIPVYSRKPFRLGEHLSRLQHSLDGIRLQNPHTEEQWAGLIERIIELNEGDDQYLYLHITRGVAKRDHAFPREVTPTVFIMSNPLPAPPAKLLVSGVSAITARDNRWGRCDIKAISLLPNILLRQLAVDAQAMETILLRDGLLTEGAASNIFIVKDDLLLTPPKDHRILPGITYDVVLELAETHGVPHATREISELELRTAREIMLTSSTKEILPITQLDGQPIGNGTPGPVFQQLDRLYQAYKLEVMRGHAPRQ

SEQ ID NO: 35 - DNA, 1257 bp, Neisseria gonorrhoeae
atgaggataaatatgaaccgtaacgaaattttattcgaccgcgccaaggccatcatccccggcggcgtgaattcgcccgtgcgcgcattcggcgtcggcggcgtgccgcgcttcatcaaaaaagccgaaggcgcgtattgggacgaaaacggcacgcgctacaccgattatgtcggctcttgggggcctgcgattgtcggacacgcgcatcccgaagtcgtcgaagccgtggaagctgcgttgggcggtttgtcgttcggcgcgcccaccgaaggcgaaatcgccattgccgaacaaattgccgaaattatgccgtctgtcgaacggctgcgcctcgtcagctccggcacggaagcgacgatgactgccatccgtctggcacgcggttttaccggccgcgacaaaatcatcaaatttgaaggctgctaccacggccattccgacagcctgttggtgaaagcaggcagcctgcttaccttcggcaatccttcttccgccggtgtgcctgccgacaccaaacatactttggtactcgaatacaacaacatcgcccaactcgaagcctttgcccaaagcggcgacgaaatcgcctgcgtgattgtcgaacccttcgtcggcaatatgaacctcgtccgcccgaccgaagcctttgtcaaagccttgcgcggattgaccgaaaaacacggcgcggtgttgatttacgacgaagtgatgaccggtttccgcgtcgcgctcggcggcgcgcagctgcacggcatcacgcccgacctgaccacgatgggcaaagtcatcggcggtatgccgcttgccgcgttcggcggacgcaaagacatcatgtgtatttccccgttgggcggcgtgtatcaggcaggtacattatcaggcaacccgattgccgtcgccgccggcttgaaaacgctggaaatcatccgcgaaggcttctatgaaaacctgaccgccttgacacaacgccttaacggtattgccgccgccaaagcgcacggtatcgagtttgccgccgacagcgtgggcggtatgttcggtctgtatttcgccgcacacgtgccgcgaaactatgccgatatggcgcgctccaatatcgacgctttcaaacgcttcttccacggcatgctcgaccgcggcattgccttcggcccgtccgcttatgaagcgggtttcgtttccgccgcgcatacgcccgagctgattgacacggttgcggttgcggttgaagtgttcaaggcgatggctgcatga

SEQ ID NO: 36 - protein, 430 aa, Neisseria gonorrhoeae
MRINMNRNEILFDRAKAIIPGGVNSPVRAFGSVGGVPRFIKKAEGAYVWDENGTRYTDYVGSWGPAIVGHAHPEVVEAVREAALGGLSFGAPTEGEIAIAEQIAEIMPSVERLRLVSSGTEATMTAIRLARGFTGRDKIIKFEGCYHGHSDSLLVKAGSGLLTFGNPSSAGVPADFTKHTLVLEYNNIAQLEEAFAQSGDEIACVIVEPFVGNMNLVRPTEAFVKALRGLTEKHGAVLIYDEVMTGFRVALGGAQSLHGITPDLTTMGKVIGGGMPLAAFGGRKDIMECISPLGGVYQAGTLSGNPIAVAAGLKTLEIIQREGFYENLTALTQRLANGIAAAKAHGIEFAADSVGGMFGLYFAAHVPRNYADMARSNIDAFKRFFHGMLDRGIAFGPSAYEAGFVSAAHTPELIDETVAVAVEVFKAMAA

SEQ ID NO: 37 - DNA, 915 bp, Pseudomonas aeruginosa
atgtcgatggccgatcgtgatggcgtgatctggtatgacggtgaactggtgcagtggcgcgacgcgaccacgcacgtgctgacccataccctgcactatggaatgggcgtgttcgagggcgtgcgcgcctacgacaccccgcagggcacggcgatcttccgcctgcaggcgcataccgaccggctgttcgactccgcgcacatcatgaacatgcagatcccgtacagccgcgacgagatcaacgaggcgacccgcgccgccgtgcgcgagaacaacctggaaagcgcctatatccgcccgatggtgttctacggaagcgaaggcatgggcctgcgcgccagcggcctgaaggtccatgtgatcatcgccgcctggagctggggcgcctacatgggcgaggaagccctgcagcaaggcatcaaggtgcgcagttccttcacccgccaccacgtcaacatctcgatgacccgcgccaagtccaacggcgcctacatcaactcgatgctggccctccaggaagcgatctccggcggcgccgacgaggccatgatgctcgatccggaaggctacgtggccgaaggctccggcgagaacatcttcatcatcaaggatggcgtgatctacaccccggaagtcaccgcctgcctgaacggcatcactcgtaacactatcctgaccctggccgccgaacacggttttaaactggtcgagaagatcacccgcgacgaggtgtacatcgccgacgaggccttcttcactggcactgccgcggaagtcacgccgatccgcgaagtggacggtcgcaagatcggcgccggccgccgtggcccggtcaccgaaaagctgcagaaagccttcgacctggtcagcggcaagaccgaggcccacgccgagtggcgtaccctggtcaagtaa

SEQ ID NO: 38 - protein, 307 aa, Pseudomonas aeruginosa
MSMADRDGVIWYDGELVQWRDATTHVLTHTLHYGMGVFEGVRAYDTPQGTAIFRLQAHTDRLFDSAHIMNMQIPYSRDEINEATRAAVRENNLESAYIRPMVFYGSEGMGLRASGLKVHVIIAAWSWGAYMGEEALQQGIKVRTSSFTRHHVNISMTRAKSNGAYINSMLALQEAISGGADEAMMLDPEGYVAEGSGENIFIIKDGVIYTPEVTACLNGITRNTILTLAAEHGFKLVEKRITRDEVYIADEAFFTGTAAEVTPIREVDGRKIGAGRRGPVTEKLQKAYFDLVSGKTEAHAEWRTLVK

SEQ ID NO: 39 - DNA, 1398 bp, Rhodopseudomonas palustris
atgaagctgataccgtgccgcgcctttcaccccccggccgcgcagtgcatgaggagcgccatgttagacaagatcaagcccacgtccgccgtcaacgcgccgaacgatctcaacgcgttctggatgccgttcaccgcgaaccgggccttcaagcgcgcgccgaagatggtcgtgggtgccgaaggcatgcactacatcaccgccgatggtcgcaagatcatcgacgccgcctcgggcatgtggtgcaccaatgcgggccatggccgcaaggaaatcgccgaggcgatcaaggcgcaggccgatgaactcgacttctcgccgccgttccagttccagccgaaggcgttcgaactcgccagccggatcgccgatctggcgccggaaggcctcgatcacgtgttcttctgcaattcgggctcggaagccggcgacaccgcgctgaagatcgcggtcgcctatcagcagatcaagggcggctcacgcacccgcctgatcggccgcgagcgcggctatcacggcggcttcggcggcaccgcggtcggcggcatcggcaacaaccgcaagatgttcggtccgctgctcaacggcgtcgatcatctgcctgcgacttatgatcgcgacaagcaggctttcaccatcggcgagccggaatacggcgcgcacttcgccgaagcgcttgaaggcctcgtcaatctgcacggcgccaacaccatcgcggcggtgatcgtcgagccgatggccggctccaccggcgtgctgccggcgccgaagggctatctcaagaagctgcgcgagatcaccaagaagcacggcatcctgctgatcttcgacgaggtcatcaccggctacggccgtctcggctatgccttcgcgtccgaacgttacggcgtcaccccggacatgatcaccttcgccaagggcgtcaccaatggtgcggtgccgatgggcggcgtgatcacctcggcggagatccacgatgcgttcatgaccggccccgagcacgcggtcgagctggcgcacggctacacctattcggcgcatccgctcgcctgcgcggccggcatcgccaccctcgacatctaccgcgacgagaagctgttcgagcgcgccaaggcgctggagccgaagtttgccgaggcggtgatgtcgctgaagtcggccccgaacgtggtcgacatccgcaccgtcggcctgacggcgggtatcgacctcgcttcgatcgccgatgcggtcggcaagcgtggcttcgaagcgatgaatgccggcttccacgaccacgagctgatgctgcggatcgccggcgacaccctggcgctgaccccgccgctgatcctcagcgaggaccacatcggtgagatcgtcgacaaggtcggcaaggtgatccgcgcggtcgcctga

SEQ ID NO: 40 - protein, 468 aa, Rhodopseudomonas palustris
MKLIPCRAFHPPAAQCMRSAMLDKIKPTSAVNAPNDLNAFWMPFTANRAFKRAPKMVVGAEGMHYITADGRKIIDAASGMWCTNAGHGRKEIAEAIKAQADELDFSPPFQFGQPKAFELASRIADLAPEGLDHVFFCNSGSEAGDTALKIAVAYQQIKGQGSRTRLIGRERGYHGVGFGGTAVGGIGNNRKMFGPLLNGVDHLPATYDRDKQAFTIGEPEYGAHFAEALEGLVNLHGANTIAAVIVEPMAGSTGVLPAPKGYLKKLREITKKHGILLIFDEVITGYGRLGYAFASERYGVTPDMITFAKGVTNGAVPMGGVITSAEIHDAFMTGPEHAVELAHGYTYSAHPLACAAGIATLDIYRDEKLFERAKALEPKFAEAVMSLKSAPNVVDIRTVGLTAGIDLASIADAVGKRGFEAMNAGFHDHELMLRIAGDTLALTPPLILSEDHIGEIVDKVGKVIRAVA

SEQ ID NO: 41 - DNA, 1353 bp, Bacillus subtilis
atggagatgatggggatggaaaacattcagcaaaatcagggattaaagcaaaaagatgagcaatttgtgtggcatgccatgaagggagcgcatcaagcggacagcctgatagcccagaaggccgaaggggcctgggtaaccgacacagacggacgccgctatttggatgcgatgtccggtttgtggtgcgtcaacattggttacggcagaaaggagcttgcggaggctgcctatgagcaactaaaggagctgccttactacccgttaacgcaaagtcacgcacccgcaattcaactggcggaaaagctgaatgaatggcttggcggcgattatgttatttttttttccaacagcggatcggaagcaaacgaaactgcttttaaaattgcccgccagtaccatctgcaaaacggcgaccacagccgttataaattcatctcaagatatcgggcataccacggcaatacattgggagcgctctcagctaccggacaggcgcagcggaaatataaatacgagcctttgagccaagggttcctgcatgcagctccgccagatatataccggaatcctgatgatgcagacacgcttgaaagcgcaaatgaaatcgaccgcatcatgacatgggaattaagcgaaacgattgccggggtcattatggagcccatcattacaggcggaggcatcctaatgccgccggacggatatatgaagaaggtggaggacatttgccggcgccacggagcccttttgatttgcgatgaagtgatctgcgggtttggacggacaggtgagccgttcgggtttatgcactacggtgtgaagcctgatatcattacgatggcaaagggaatcacaagcgcgtatctgccattgtcagcgactgctgtgaaacgggacattttcgaagcgtatcagggggaagctccttatgaccgtttccgccacgtgaacacgttcggcggaagcccggctgcctgtgctttggcgttgaaaaacctgcaaattatggaggacgaacagctgattcagcgatcccgtgatcttggagcaaagcttttaggtgagcttcaagctctgagagaacacccggcagtcggggatgttagaggaaaagggctgctgatcggaatcgaactcgtcaaagacaaattgactaaagagccggctgatgccgccaaagtaaaccaagtggttgcggcgtgcaaagaaaaagggctgatcatcggcaaaaacggcgatacagtcgccggctacaacaatgtcatccacgttgcgccgccattttgcctgacagaagaggacctttcctttatcgtgaaaacggtgaaagaaagctttcaaacgatataa

SEQ ID NO: 42 - protein, 450 aa, Bacillus subtilis
MEMMGMENIQQNQGLKQKDEQFVWHAMKGAHQADSLIAQKAEGAWVTDTDGRRYLDAMSGLWCVNIGYGRKELAEAAYEQLKELPYYPLTQSHAPAIQLAEKLNEWLGGDYVIFFSNSGSEANETAFKIARQYHLQNGDHSRYKFISRYRAYHGNTLGALSATGQAQRKYKYEPLSQGFLHAAPPDIYRNPDDADTLESANEIDRIMTWELSETIAGVIMEPIITGGGILMPPDGYMKKVEDICRRHGALLICDEVICGFGRTGEPFGFMHYGVKPDIITMAKGITSAYLPLSATAVKRDIFEAYQGEAPYDRFRHVNTFGGSPAACALALKNLQIMEDEQLIQRSRDLGAKLLGELQALREHPAVGDVRGKGLLIGIELVKDKLTKEPADAAKVNQVVAACKEKGLIIGKNGDTVAGYNNVIHVAPPFCLTEEDLSFIVKTVKESFQTI

SEQ ID NO: 43 - DNA, 1407 bp, Pseudomonas aeruginosa
atgaacgcaagactgcacgccacgtcccccctcggcgacgccgacctggtccgtgccgaccaggcccactacatgcacggctaccacgtgttcgacgaccaccgcgtcaacggctcgctgaacatcgccgccggcgacggcgcctatatctacgacaccgccggcaaccgctacctcgacgcggtgggcggcatgtggtgcaccaacatcggcctggggcgcgaggaaatggctcgcaccgtggccgagcagacccgcctgctggcctattccaatcccttctgcgacatggccaacccgcgcgccatcgaactctgccgcaagctcgccgagctggcccccggcgacctcgaccacgtgttcctcaccaccggcggttccaccgccgtggacaccgcgatccgcctcatgcactactaccagaactgccgcggcaagcgcgccaagaagcacgtcatcacgcggatcaacgcctaccacggctcgaccttcctcggcatgtcgctgggcggcaagagcgccgaccggccggccgagttcgacttcctcgacgagcgcatccaccacctcgcctgtccctattactaccgcgctccggaagggctgggcgaagccgagttcctcgatggcctggtggacgagttcgaacgcaagatcctcgaactgggcgccgaccgggtgggggcgttcatctccgagccggtgttcggctccggcggcgtgatcgtcccgcccgcgggctaccacaggcggatgtgggagctgtgccagcgctacgacgtgctgtacatctccgacgaagtggtgacctccttcggccgcctcggccacttcttcgccagccaggcggtgttcggcgtacagccggacatcatcctcaccgccaagggcctcacctccggctaccagccgctgggcgcgtgcatcttctcccggcgcatctgggaggtgatcgccgagccggacaagggccgctgcttcagccatggtttcacctactccggccacccggtggcctgcgcggcggcgctgaagaacatcgagatcatcgagcgcgagggcttgctcgcccacgccgacgaggtcggccgctacttcgaggagcgcctgcaaagcctccgcgacctgcccatcgtcggcgacgtgcgcgggatgcgcttcatggcctgtgtcgagttcgtcgccgacaaggcgagcaaggcgctgtttccggaaagcctgaacatcggcgagtgggtccacctgcgggcgcagaagcgcggcctgctggttcgtccgatcgtccacctgaacgtgatgtcgccgccgctgatcctcacccgcgaacaggtcgataccgtggtccgggtgctgcgcgagagcatcgaggaaaccgtggaggatcttgtccgcgccggtcaccggtaa

SEQ ID NO: 44 - protein, 468 aa, Pseudomonas aeruginosa
MNARLHATSPLGDADLVRADQAHYMHGYHVFDDHRVNGSLNIAAGDGAYIYDTAGNRYLDAVGGMWCTNIGLGREEMARTVAEQTRLLAYSNPFCDMANPRAIELCRKLAELAPGDLDHVFLTTGGSTAVDTAIRLMHYYQNCRGKRAKKHVITRINAYHGSTFLGMSLGGKSADRPAEFDFLDERIHHLACPYYYRAPEGLGEAEFLDGLVDEFERKILELGADRVGAFISEPVFGSGGVIVPPAGYHRRMWELCQRYDVLYISDEVVTSFGRLGHFFASQAVFGVQPDIILTAKGLTSGYQPLGACIFSRRIWEVIAEPDKGRCFSHGFTYSGHPVACAAALKNIEIIEREGLLAHADEVGRYFEERLQSLRDLPIVGDVRGMRFMACVEFVADKASKALFPESLNIGEWVHLRAQKRGLLVRPIVHLNVMSPPLILTREQVDTVVRVLRESIEETVEDLVRAGHR

SEQ ID NO: 45 - DNA, 1335 bp, Pseudomonas aeruginosa
atgacaatgaatgacgagccgcagtcgagcagcctcgacaacttctggatgcccttcaccgccaaccgccagttcaaggcgcggccgcgcctgctggaaagcgccgaaggcatccactatatcgcccagggcgggcgccgcatcctcgacggcaccgccggcctctggtgctgcaatgccggccacggccggcgcgagatcagcgaagcggtggcccggcagatcgccaccctcgactacgccccgccgttccagatgggtcacccgctgccgttcgaactcgccgcgcggctgacggaaatcgccccgccgagcctgaacaaagtattcttcaccaactccggctcggaatcggcggacaccgcgctgaagatcgcccttgcctaccagcgcgccatcggccagggcacccgcacccgcctgatcggccgcgaactgggctaccacggggtcggcttcggcggcctgtcggtaggcggtatggtcaacaaccgcaaggccttctccgccaacctgctgccgggggtcgaccacctgccgcacaccctggacgtcgcccgcaacgccttcaccgtcggcctgcccgagcatggcgtggaaaaggccgaggagctggaacgcctggtgaccctgcacggcgccgagaatatcgccgcggtgatcgtcgagccgatgtccggctcggccggcgtggtgctgccgcccaagggctaccttcagcggctgcgcgagataacccgcaagcatggcatcctgctgatcttcgacgaagtgatcaccggtttcggccgcgtcggcgaagccttcgccgcgcagcgctggggcgtcgtcccggacctgctgacctgcgccaaggggctgaccaacggcagcatcccgatgggcgccgtattcgtcgacgagaagatccatgctgccttcatgcaaggcccgcagggcgccatcgagttcttccacggctatacctattccggccatccggtagcctgcgccgccgccctggcgaccctggacatctaccgtcgcgacgacctgttccagcgggccgtcgaactggaaggctactggcaggacgcgctgttcagcctgcgcgacctgcccaacgtggtcgacatccgcgccgtaggcctggtcggcggcgtgcaactggcgccgcacgcggacggccccggcaagcgcggctacgacgtcttcgagcgctgcttctgggagcacgacctgatggtccgggtgaccggcgacatcatcgccatgtcgccgccgctgatcatcgacaagccccacatcgaccagatcgtcgagcgcctggcccaggccatccgcgccagcgtctga

SEQ ID NO: 46 - protein, 444 aa, Pseudomonas aeruginosa
MTMNDEPQSSSLDNFWMPFTANRQFKARPRLLESAEGIHYIAQGGRRILDGTAGLWCCNAGHGRREISEAVARQIATLDYAPPFQMGHPLPFELAARLTEIAPPSLNKVFFTNSGSESADTALKIALAYQRAIGQGTRTRLIGRELGYHGVGFGGLSVGGMVNNRKAFSANLLPGVDHLPHTLDVARNAFTVGLPEHGVEKAEELERLVTLHGAENIAAVIVEPMSGSAGVVLPPKGYLQRLREITRKHGILLIFDEVITGFGRVGEAFAAQRWGVVPDLLTCAKGLTNGSIPMGAVFVDEKIHAAFMQGPQGAIEFFHGYTYSGHPVACAAALATLDIYRRDDLFQRAVELEGYWQDALFSLRDLPNVVDIRAVGLVGGVQLAPHADGPGKRGYDVFERCFWEHDLMVRVTGDIIAMSPPLIIDKPHIDQIVERLAQAIRASV

(19) United States
(12) Patent Application Publication
(10) Pub. No.: US 2013/0237698 A1
(43) Pub. Date: Sep. 12, 2013

(54) PREPARATION OF 6-AMINOCAPROIC ACID FROM ALPHA-KETOPIMELIC ACID

(71) Applicants: Stefanus Cornelis Hendrikus Turk, Echt (NL); Martin Schürmann, Echt (NL); Axel Christoph Trefzer, Echt (NL); Petronella Catharina Raemakers-Franken, Echt (NL); Hildegard Henna Menke, Echt (NL)

(72) Inventors: Stefanus Cornelis Hendrikus Turk, Echt (NL); Martin Schürmann, Echt (NL); Axel Christoph Trefzer, Echt (NL); Petronella Catharina Raemakers-Franken, Echt (NL); Hildegard Henna Menke, Echt (NL)

(73) Assignee: DSM IP ASSETS B.V., Heerlen (NL)

(21) Appl. No.: 13/821,791

(22) PCT Filed: Aug. 26, 2011
(86) PCT No.: PCT/EP11/64720

§ 371 Date: May 28, 2013

(30) Foreign Application Priority Data
Sep. 10, 2010 (EP) 10176131.0

(51) Int. Cl. C12P 13/00; C07D 223/10

(52) U.S. Cl. CPC C12P 13/001; C07D 223/10
USPC 536/23.2; 540/538; 435/128; 435/252.33

(57) ABSTRACT

The invention is directed to a method for preparing 6-aminocaproic acid, comprising decarboxylating alpha-aminopimelic acid, using at least one biocatalyst comprising an enzyme having alpha-aminopimelic acid decarboxylase activity. The invention is further directed to a method for preparing caprolactam from 6-aminocaproic acid prepared by said method, to a host cell suitable for use in a method according to the invention and to a polynucleotide encoding a decarboxylase that may be used in a method according to the invention.

The invention relates to a method for preparing 6-aminocaproic acid (hereinafter also referred to as ‘6-ACA’). The invention further relates to a method for preparing ε-caprolactam (hereafter referred to as ‘caprolactam’) from 6-ACA. The invention further relates to a host cell which may be used in the preparation of 6-ACA or caprolactam.

Caprolactam is a lactam which may be used for the production of polyamide, for instance nylon-6 or nylon-6,12 (a copolymer of caprolactam and laurolactam). Various manners of preparing caprolactam from bulk chemicals are known in the art and include the preparation of caprolactam from cyclohexanone, toluene, phenol, cyclohexanol, benzene or cyclohexane. These intermediate compounds are generally obtained from mineral oil. In view of a growing desire to prepare materials using more sustainable technology it would be desirable to provide a method wherein caprolactam is prepared from an intermediate compound that can be obtained from a biologically renewable source or at least from an intermediate compound that is converted into caprolactam using a biochemical method. Further, it would be desirable to provide a method that requires less energy than conventional chemical processes making use of bulk chemicals from petrochemical origin.

It is known to prepare caprolactam from 6-ACA, e.g. as described in U.S. Pat. No. 6,194,572. As disclosed in WO 2005/068643, 6-ACA may be prepared biochemically by converting 6-aminohex-2-enoic acid (6-AHEA) in the presence of an enzyme having α,β enoate reductase activity. The 6-AHEA may be prepared from lysine, e.g. biochemically or by pure chemical synthesis. Although the preparation of 6-ACA via the reduction of 6-AHEA is feasible by the methods disclosed in WO 2005/068643, the inventors have found that—under the reduction reaction conditions—6-AHEA may spontaneously and substantially irreversibly cyclise to form an undesired side-product, notably β-homoproline. This cyclisation may be a bottleneck in the production of 6-ACA, and may lead to a considerable loss in yield.

WO 2009/113855 discloses new reaction pathways for the preparation of 6-ACA, namely the preparation of 6-ACA from alpha-ketopimelic acid (AKP) via the intermediate 5-formylpentanoic acid (a.k.a. 5-formyl valeric acid, 5-FVA) or via the intermediate alpha-aminopimelic acid (AAP). WO 2009/113855 also discloses biocatalysts capable of catalysing at least one of the reaction steps in the preparation of 6-ACA from AKP. Although WO 2009/113855 discloses methods that are effective in producing 6-ACA, it would be desirable to increase the production rate of biocatalytically produced 6-ACA, in particular in a method wherein 6-ACA is fully biocatalytically produced from AKP.

It is an object of the invention to provide a novel method for preparing 6-ACA or caprolactam—which may, inter alia, be used for the preparation of polyamide—or an intermediate compound for the preparation of 6-ACA or caprolactam, that can serve as an alternative for known methods.

It is a further object to provide a novel method that would overcome one or more of the drawbacks of the above mentioned prior art.

One or more further objects which may be solved in accordance with the invention, will follow from the description, below.

It has now been found possible to prepare 6-ACA from AAP using a specific bioacatalyst having alpha-aminopimelic acid decarboxylase activity. Accordingly, the present invention relates to a method for preparing 6-aminocaproic acid, comprising decarboxylating alpha-aminopimelic acid, using at least one biocatalyst comprising an enzyme having alpha-aminopimelic acid decarboxylase activity, wherein said enzyme comprises an amino acid sequence represented by any of the SEQUENCE ID NO's: 2, 5, 8 and 11 and homologues having alpha-aminopimelic acid decarboxylase activity of said sequences.

In an embodiment, 6-ACA prepared in a method of the invention is used for preparing caprolactam. Such method comprises cyclising the 6-amino-caproic acid, optionally in the presence of a biocatalyst.

In accordance with the invention, no problems have been noticed with respect to an undesired cyclisation of an intermediate product, when forming 6-ACA and optionally caprolactam, resulting in a loss of yield.

It is envisaged that a method of the invention allows a yield comparable to or even better than the method described in WO 2005/68643. It is envisaged that a method of the invention may in particular be favourable if use is made of a living organism—in particular in a method wherein growth and maintenance of the organism is taken into account.

It is further envisaged that in an embodiment of the invention the productivity of 6-ACA (g/l.h formed) in a method of the invention is improved.

The term “or” as used herein is defined as “and/or” unless specified otherwise.

The term “a” or “an” as used herein is defined as “at least one” unless specified otherwise.

When referring to a noun (e.g. a compound, an additive, etc.) in the singular, the plural is meant to be included.

When referring herein to carboxylic acids or carboxylates, e.g. 6-ACA,

AAP, another amino acid, or AKP, these terms are meant to include the protonated carboxylic acid group (i.e. the neutral group), their corresponding carboxylate (their conjugated bases) as well as salts thereof. When referring herein to amino acids, e.g. 6-ACA, this term is meant to include amino acids in their zwitterionic form (in which the amino group is in the protonated and the carboxylate group is in the deprotonated form), the amino acid in which the amino group is protonated and the carboxylic group is in its neutral form, and the amino acid in which the amino group is in its neutral form and the carboxylate group is in the deprotonated form, as well as salts thereof.

When referring to a compound of which stereoisomers exist, the compound may be any of such stereoisomers or a combination thereof. Thus, when referred to, e.g., an amino acid of which enantiomers exist, the amino acid may be the L-enantiomer, the D-enantiomer or a combination thereof. In case a natural stereoisomer exists, the compound is preferably a natural stereoisomer.

When an enzyme is mentioned with reference to an enzyme class (EC) between brackets, the enzyme class is a class wherein the enzyme is classified or may be classified, on the basis of the Enzyme Nomenclature provided by the Nomenclature Committee of the International Union of Biochemistry and Molecular Biology (NC-IUBMB), which nomenclature may be found at http://www.chem.qmul.ac.uk/iubmb/enzyme/. Other suitable enzymes that have not (yet) been classified in a specified class but may be classified as such, are meant to be included.

The term “homologue” is used herein in particular for polynucleotides or polypeptides having a sequence identity of at least 40%, more preferably at least 60%, more preferably at least 65%, more preferably at least 70%, more preferably at least 75%, more preferably at least 80%, in particular at least 85%, more in particular at least 90%, at least 91%, at least 92%, at least 93%, at least 94%, at least 95%, at least 96%, at least 97%, at least 98% or at least 99%.

Further, homologues usually have a significant sequence similarity, usually of more than 30%, in particular a sequence similarity of at least 35%, preferably at least 40%, more preferably at least 60%, more preferably at least 65%, more preferably at least 70%, more preferably at least 75%, more preferably at least 80%, in particular at least 85%, more in particular at least 90%, at least 91%, at least 92%, at least 93%, at least 94%, at least 95%, at least 96%, at least 97%, at least 98% or at least 99%.

Homologues generally have an intended function in common with the polynucleotide respectively polypeptide of which it is a homologue, such as encoding the same peptide respectively being capable of catalysing the same reaction (typically the conversion of the same substrate into the same compound) or a similar reaction. A ‘similar reaction’ typically is a reaction of the same type, e.g. a decarboxylation, an aminotransfer, a C1-elongation. Accordingly, as a rule of thumb, homologous enzymes can be classified in an EC class sharing the first three numerals of the EC class (x.y.z), for example EC 4.1.1 for carboxylyases. Typically, in the similar reaction, a substrate of the same class (e.g. an amine, a carboxylic acid, an amino acid) as the substrate for the reaction to which the similar reaction is similar is converted into a product of the same class as the product of the reaction to which the similar reaction is similar. Similar reactions in particular include reactions that are defined by the same chemical conversion as defined by the same KEGG RDM patterns, wherein the R-atoms and D-atoms describe the chemical conversion (KEGG RDM patterns: Oh, M. et al. (2007) Systematic analysis of enzyme-catalyzed reaction patterns and prediction of microbial biodegradation pathways. J. Chem. Inf. Model., 47, 1702-1712).

The term homologue is also meant to include nucleic acid sequences (polynucleotide sequences) which differ from another nucleic acid sequence due to the degeneracy or experimental adaptation of the genetic code and encode the same polypeptide sequence.

The term “functional analogue” is used herein for nucleic acid sequences that differ from a given sequence of which said analogue is an analogue, yet that encode a peptide (protein, enzyme) having the same amino acid sequence or that encode a homologue of such peptide. In particular, preferred functional analogues are nucleotide sequences having a similar, the same or a better level of expression in a host cell of interest as the nucleotide sequence of which it is referred to as being a functional analogue of. In this respect it is observed that, as the skilled person understands, a better level of expression usually is a higher level of expression if the expression of the peptide (protein, enzyme) is desired. However, in specific embodiment a better level of expression may be a lower expression level since this might be desirable in context of a metabolic pathway in said host cell. The functional analogue can be a naturally occurring sequence, i.e. a wild-type functional analogue, or a genetically modified sequence, i.e. a non-wild type functional analogue. Codon optimised sequences encoding a specific peptide, are generally non-wild type functional analogues of a wild-type sequence, designed to achieve a desired expression level.

In particular, preferred functional analogues are nucleotide sequences having a similar, the same or a better level of expression in a host cell of interest as the nucleotide sequence of which it is referred to as being a functional analogue of.

Sequence identity or similarity is herein defined as a relationship between two or more polypeptide sequences or two or more nucleic acid sequences, as determined by comparing the sequences. Usually, Sequence Identities or similarities are compared over the whole length of the sequences, but may however also be compared only for a part of the sequences aligning with each other. In the art, “identity” or “similarity” also means the degree of sequence relatedness between polypeptide sequences or nucleic acid sequences, as the case may be, as determined by the match between such sequences. Preferred methods to determine identity or similarity are designed to give the largest match between the sequences tested. In context of this invention a preferred computer program method to determine identity and similarity between two sequences includes BLASTP and BLASTN (Altschul, S. F. et al., J. Mol. Biol. 1990, 215, 403-410, publicly available from NCBI and other sources (BLAST Manual, Altschul, S., et al., NCBI NLM NIH Bethesda, Md. 20894). Preferred parameters for polypeptide sequence comparison using BLASTP are gap open 10.0, gap extend 0.5, Blosum 62 matrix. Preferred parameters for nucleic acid sequence comparison using BLASTN are gap open 10.0, gap extend 0.5, DNA full matrix (DNA identity matrix).

In accordance with the invention, a biocatalyst is used, i.e. at least one reaction step in the method is catalysed by a biological material or moiety derived from a biological source, for instance an organism or a biomolecule derived there from. The biocatalyst may in particular comprise one or more enzymes. The biocatalyst may be used in any form. In an embodiment, one or more enzymes are used isolated from the natural environment (isolated from the organism it has been produced in), for instance as a solution, an emulsion, a dispersion, (a suspension of) freeze-dried cells, as a lysate, or immobilised on a support. In an embodiment, one or more enzymes form part of a living organism (such as living whole cells).

The enzymes may perform a catalytic function inside the cell. It is also possible that the enzyme may be secreted into a medium, wherein the cells are present.

Living cells may be growing cells, resting or dormant cells (e.g. spores) or cells in a stationary phase. It is also possible to use an enzyme forming part of a permeabilised cell (i.e. made permeable to a substrate for the enzyme or a precursor for a substrate for the enzyme or enzymes).

A biocatalyst used in a method of the invention may in principle be any organism, or be obtained or derived from any organism. The organism may be eukaryotic or prokaryotic. In particular the organism may be selected from animals (including humans), plants, bacteria, archaea, yeasts and fungi.

In an embodiment a biocatalyst originates from an animal, in particular from a part thereof—e.g. liver, pancreas, brain, kidney, heart or other organ. The animal may in particular be selected from the group of mammals, more in particular selected from the group of Leporidae, Muridae, Suidae and Bovidae.

Suitable plants in particular include plants selected from the group of Asplenium; Cucurbitaceae, in particular Curcurbita, e.g. Curcurbita moschata (squash), or Cucumis; Mercurialis, e.g. Mercurialis perennis; Hydnocarpus; and Ceratonia.

Suitable bacteria may in particular be selected amongst the group of Vibrio, Pseudomonas, Bacillus, Corynebacterium, Brevibacterium, Enterococcus, Streptococcus, Klebsiella, Lactococcus, Lactobacillus, Clostridium, Escherichia, Thermus, Mycobacterium, Zymomonas, Proteus, Agrobacterium, Geobacillus, Acinetobacter, Ralstonia, Rhodobacter, Paracoccus, Novosphingobium, Nitrosomonas, Legionella, Neisseria, Rhodopseudomonas, Staphylococcus, Thermotoga Deinococcus and Salmonella.

Suitable archaea may in particular be selected amongst the group of Archaeoglobus, Aeropyrum, Halobacterium, Methanosarcina, Methanococcus, Thermoplasma, Pyrobaculum, Methanocaldococcus, Methanobacterium, Methanosphaera, Methanopyrus and Methanobrevibacter.

Suitable fungi may in particular be selected amongst the group of Rhizopus, Neurospora, Penicillium and Aspergillus.

A suitable yeast may in particular be selected amongst the group of Candida, Hansenula, Kluyveromyces and Saccharomyces.

It will be clear to the person skilled in the art that use can be made of a naturally occurring biocatalyst (wild type) or a mutant of a naturally occurring biocatalyst with suitable activity in a method according to the invention. Properties of a naturally occurring biocatalyst may be improved by biological techniques known to the skilled person in the art, such as e.g. molecular evolution or rational design. Mutants of wild-type biocatalysts can for example be made by modifying the encoding DNA of an organism capable of acting as a biocatalyst or capable of producing a biocatalytic moiety (such as an enzyme) using mutagenesis techniques known to the person skilled in the art (random mutagenesis, site-directed mutagenesis, directed evolution, gene recombination, etc.). In particular the DNA may be modified such that it encodes an enzyme that differs by at least one amino acid from the wild-type enzyme, so that it encodes an enzyme that comprises one or more amino acid substitutions, deletions and/or insertions compared to the wild-type, or such that the mutants combine sequences of two or more parent enzymes or by effecting the expression of the thus modified DNA in a suitable (host) cell. The latter may be achieved by methods known to the skilled person in the art such as codon optimisation or codon pair optimisation, e.g. based on a method as described in WO 2008/000632.

A mutant biocatalyst may have improved properties, for instance with respect to one or more of the following aspects: selectivity towards the substrate, activity, stability, solvent tolerance, pH profile, temperature profile, substrate profile, susceptibility to inhibition, cofactor utilisation and substrate-affinity. Mutants with improved properties can be identified by applying e.g. suitable high through-put screening or selection methods based on such methods known to the skilled person in the art.

When referred to a biocatalyst, in particular an enzyme, from a particular source, recombinant biocatalysts, in particular enzymes, originating from a first organism, but actually produced in a (genetically modified) second organism, are specifically meant to be included as biocatalysts, in particular enzymes, from that first organism.

AAP may be obtained in any way. In a specific embodiment, AAP is obtained by chemically converting AKP. Further, AAP can be prepared from 2-oxopimelic acid by catalytic Leuckart-Wallach reaction as described for similar compounds. This reaction is performed with ammonium formate in methanol and [RhCp*Cl₂]₂ as homogeneous catalyst (M. Kitamura, D. Lee, S. Hayashi, S. Tanaka, M. Yoshimura J. Org. Chem. 2002, 67, 8685-8687). Alternatively, the Leuckart-Wallach reaction can be performed with aqueous ammonium formate using [Ir^{III}Cp*(bpy)H₂O]SO₄ as catalyst as described by S. Ogo, K. Uehara and S. Fukuzumi in J. Am. Chem. Soc. 2004, 126, 3020-3021. Transformation of α-keto acids into (enantiomerically enriched) amino acids is also possible by reaction with (chiral) benzylamines and subsequent hydrogenation of the intermediate imine over Pd/C or Pd(OH)₂/C. See for example, R. G. Hiskey, R. C. Northrop J. Am. Chem. Soc. 1961, 83, 4798.

In a preferred method of the invention, the preparation of 6-ACA comprises an enzymatic reaction in the presence of an enzyme capable of catalysing a transamination reaction in the presence of an amino donor, selected from the group of aminotransferases (E.C. 2.6.1).

In a specific embodiment, AAP is obtained by biocatalytically converting AKP into AAP which conversion is catalysed by an aminotransferase (E.C. 2.6.1), an amino acid dehydrogenase, or another biocatalyst capable of catalysing the conversion of AKP into AAP. In general, such biocatalyst has alpha-aminopimelate 2-aminotransferase activity or alpha-aminopimelate 2-aminodehydrogenase activity.

The aminotransferase may in particular be selected amongst the group of β-aminoisobutyrate: α-ketoglutarate aminotransferases, β-alanine aminotransferases, aspartate aminotransferases, 4-amino-butyrate aminotransferases (EC 2.6.1.19), L-lysine 6-aminotransferase (EC 2.6.1.36), 2-aminoadipate aminotransferases (EC 2.6.1.39), 5-aminovalerate aminotransferases (EC 2.6.1.48), 2-aminohexanoate aminotransferases (EC 2.6.1.67) and lysine:pyruvate 6-aminotransferases (EC 2.6.1.71). In an embodiment an aminotransferase is selected amongst the group of alanine aminotransferases (EC 2.6.1.2), leucine aminotransferases (EC 2.6.1.6), alanine-oxo-acid aminotransferases (EC 2.6.1.12), β-alanine-pyruvate aminotransferases (EC 2.6.1.18), (S)-3-amino-2-methylpropionate aminotransferases (EC 2.6.1.22), L,L-diaminopimelate aminotransferase (EC 2.6.1.83).

The aminotransferase may in particular be selected amongst aminotransferases from a mammal; Mercurialis, in particular Mercurialis perennis, more in particular shoots of Mercurialis perennis; Asplenium, more in particular Asplenium unilaterale or Asplenium septentrionale; Ceratonia, more in particular Ceratonia siliqua; Rhodobacter, in particular Rhodobacter sphaeroides, Staphylococcus, in particular Staphylococcus aureus; Vibrio, in particular Vibrio fluvialis; Pseudomonas, in particular Pseudomonas aeruginosa; Rhodopseusomonas; Bacillus, in particular Bacillus weihenstephanensis and Bacillus subtilis; Legionella; Nitrosomas; Neisseria; or yeast, in particular Saccharomyces cerevisiae.

In case the enzyme is of a mammal, it may in particular originate from mammalian kidney, from mammalian liver, from mammalian heart or from mammalian brain. For instance a suitable enzyme may be selected amongst the group of β-aminoisobutyrate: α-ketoglutarate aminotransferase from mammalian kidney, in particular β-aminosobutyrate: α-ketoglutarate aminotransferase from hog kidney; β-alanine aminotransferase from mammalian liver, in particular β-alanine aminotransferase from rabbit liver; aspartate aminotransferase from mammalian heart; in particular aspartate aminotransferase from pig heart; 4-amino-butyrate aminotransferase from mammalian liver, in particular 4-amino-butyrate aminotransferase from pig liver; 4-amino-butyrate aminotransferase from mammalian brain, in particular 4-aminobutyrate aminotransferase from human, pig, or rat brain.

In an embodiment α-ketoadipate-glutamate aminotransferase from a fungus, in particular Neurospora, more in particular α-ketoadipate:glutamate aminotransferase from Neurospora crassa.

In an embodiment the aminotransferase is selected from the group of 4-amino-butyrate aminotransferase from E. coli, α-aminoadipate aminotransferase from Thermus, in particular α-aminoadipate aminotransferase from Thermus thermophilus, and 5-aminovalerate aminotransferase from Clostridium in particular from Clostridium aminovalericum.

A suitable 2-aminoadipate aminotransferase may e.g. be provided by Pyrobaculum islandicum.

In particular, the amino donor can be selected from the group of ammonia, ammonium ions, amines and amino acids. Suitable amines are primary amines and secondary amines. The amino acid may have a D- or L-configuration. Examples of amino donors are alanine, glutamate, isopropylamine, 2-aminobutane, 2-aminoheptane, phenylmethanamine, 1-phenyl-1-aminoethane, glutamine, tyrosine, phenylalanine, aspartate, β-aminoisobutyrate, β-alanine, 4-aminobutyrate, and α-aminoadipate.

In a further preferred embodiment, the method for preparing 6-ACA comprises a biocatalytic reaction in the presence of an enzyme capable of catalysing a reductive amination reaction in the presence of an ammonia source, selected from the group of oxidoreductases acting on the CH—NH₂ group of donors (EC 1.4), in particular from the group of amino acid dehydrogenases (E.C. 1.4.1). In general, a suitable amino acid dehydrogenase has 6-aminocaproic acid 6-dehydrogenase activity, catalysing the conversion of 5-FVA into 6-ACA or has α-aminopimelate 2-dehydrogenase activity, catalysing the conversion of AKP into AAP. In particular a suitable amino acid dehydrogenase be selected amongst the group of diaminopimelate dehydrogenases (EC 1.4.1.16), lysine 6-dehydrogenases (EC 1.4.1.18), glutamate dehydrogenases (EC 1.4.1.3; EC 1.4.1.4), and leucine dehydrogenases (EC 1.4.1.9).

In an embodiment, an amino acid dehydrogenase is selected amongst an amino acid dehydrogenases classified as glutamate dehydrogenases acting with NAD or NADP as acceptor (EC 1.4.1.3), glutamate dehydrogenases acting with NADP as acceptor (EC 1.4.1.4), leucine dehydrogenases (EC 1.4.1.9), diaminopimelate dehydrogenases (EC 1.4.1.16), and lysine 6-dehydrogenases (EC 1.4.1.18).

An amino acid dehydrogenase may in particular originate from an organism selected from the group of Corynebacterium, in particular Corynebacterium glutamicum; Proteus, in particular Proteus vulgaris; Agrobacterium, in particular Agrobacterium tumefaciens; Geobacillus, in particular Geobacillus stearothermophilus; Acinetobacter, in particular Acinetobacter sp. ADP1; Ralstonia, in particular Ralstonia solanacearum; Salmonella, in particular Salmonella typhimurium; Saccharomyces, in particular Saccharomyces cerevisiae; Brevibacterium, in particular Brevibacterium flavum; and Bacillus, in particular Bacillus sphaericus, Bacillus cereus or Bacillus subtilis. For instance a suitable amino acid dehydrogenase may be selected amongst diaminopimelate dehydrogenases from Bacillus, in particular Bacillus sphaericus; diaminopimelate dehydrogenases from Brevibacterium sp.; diaminopimelate dehydrogenases from Corynebacterium, in particular diaminopimelate dehydrogenases from Corynebacterium glutamicum; diaminopimelate dehydrogenases from Proteus, in particular diaminopimelate dehydrogenase from Proteus vulgaris; lysine 6-dehydrogenases from Agrobacterium, in particular Agrobacterium tumefaciens, lysine 6-dehydrogenases from Geobacillus, in particular from Geobacillus stearothermophilus; glutamate dehydrogenases acting with NADH or NADPH as cofactor (EC 1.4.1.3) from Acinetobacter, in particular glutamate dehydrogenases from Acinetobacter sp. ADP1; glutamate dehydrogenases (EC 1.4.1.3) from Ralstonia, in particular glutamate dehydrogenases from Ralstonia solanacearum; glutamate dehydrogenases acting with NADPH as cofactor (EC 1.4.1.4) from Salmonella, in particular glutamate dehydrogenases from Salmonella typhimurium; glutamate dehydrogenases (EC 1.4.1.4) from Saccharomyces, in particular glutamate dehydrogenases from Saccharomyces cerevisiae; glutamate dehydrogenases (EC 1.4.1.4) from Brevibacterium, in particular glutamate dehydrogenases from Brevibacterium flavum; and leucine dehydrogenases from Bacillus, in particular leucine dehydrogenases from Bacillus cereus or Bacillus subtilis.

In a specific embodiment the aminotransferase used for the conversion of AKP to AAP is selected from the group of aspartate aminotransferases from pig heart; α-ketoadipate:glutamate aminotransferases from Neurospora crassa or yeast; aminotransferases from shoots from Mercurialis perennis; 4-aminobutyrate aminotransferases from E. coli; α-aminoadipate aminotransferases from Thermus thermophilus; aminotransferases from Asplenium septentrionale or Asplenium unilaterale; and aminotransferases from Ceratonia siliqua.

In a specific embodiment, the aminotransferase for the conversion of AKP to AAP is selected from the group of aminotransferases from Vibrio, Pseudomonas, Bacillus, Legionella, Nitrosomonas, Neisseria, Rhodobacter, Escherichia and Rhodopseudomonas.

In particular, aminotransferases from an organism selected from the group of Bacillus subtilis, Rhodobacter sphaeroides, Legionella pneumophila, Nitrosomonas europaea, Neisseria gonorrhoeae, Pseudomonas syringae, Rhodopseudomonas palustris, Vibrio fluvialis, Escherichia coli and Pseudomonas aeruginosa, have been found suitable to catalyse the conversion of AKP to AAP.

In a specifically preferred embodiment, for the conversion of AKP to AAP an aminotransferase is used comprising an amino acid sequence according to Sequence ID NO 15, Sequence ID NO 18, Sequence ID NO 21, Sequence ID NO 23, Sequence ID NO 26, Sequence ID NO 28, Sequence ID NO 30, Sequence ID NO 32, Sequence ID NO 34, Sequence ID NO 36, Sequence ID NO 38, Sequence ID NO 40 Sequence ID NO 42, Sequence ID NO 44, Sequence ID NO 46 or a homologue of any of these sequences.

In a further embodiment, the method for preparing AAP from AKP comprises a biocatalytic reaction in the presence of an enzyme capable of catalysing a reductive amination reaction in the presence of an ammonia source, selected from the group of oxidoreductases acting on the CH—NH₂ group of donors (EC 1.4), in particular from the group of amino acid dehydrogenases (E.C. 1.4.1). In general, a suitable amino acid dehydrogenase has α-aminopimelate 2-dehydrogenase activity, catalysing the conversion of AKP into AAP.

In particular, a suitable amino acid dehydrogenase may be selected from the group of diaminopimelate dehydrogenases (EC 1.4.1.16), glutamate dehydrogenases (EC 1.4.1.3; EC 1.4.1.4), and leucine dehydrogenases (EC 1.4.1.9).

In an embodiment, an amino acid dehydrogenase is selected amongst amino acid dehydrogenases classified as glutamate dehydrogenases acting with NAD or NADP as acceptor (EC 1.4.1.3), glutamate dehydrogenases acting with NADP as acceptor (EC 1.4.1.4), leucine dehydrogenases (EC 1.4.1.9), and diaminopimelate dehydrogenases (EC 1.4.1.16).

An amino acid dehydrogenase may in particular originate from an organism selected from the group of Corynebacterium, in particular Corynebacterium glutamicum; Proteus, in particular Proteus vulgaris; Agrobacterium, in particular Agrobacterium tumefaciens; Geobacillus, in particular Geobacillus stearothermophilus; Acinetobacter, in particular Acinetobacter sp. ADP1; Ralstonia, in particular Ralstonia solanacearum; Salmonella, in particular Salmonella typhimurium; Saccharomyces, in particular Saccharomyces cerevisiae; Brevibacterium, in particular Brevibacterium flavum; and Bacillus, in particular Bacillus sphaericus, Bacillus cereus or Bacillus subtilis.

For instance, a suitable amino acid dehydrogenase may be selected amongst diaminopimelate dehydrogenases from Bacillus, in particular Bacillus sphaericus; diaminopimelate dehydrogenases from Brevibacterium sp.; diaminopimelate dehydrogenases from Corynebacterium, in particular diaminopimelate dehydrogenases from Corynebacterium glutamicum; diaminopimelate dehydrogenases from Proteus, in particular diaminopimelate dehydrogenase from Proteus vulgaris; glutamate dehydrogenases acting with NADH or NADPH as cofactor (EC 1.4.1.3) from Acinetobacter, in particular glutamate dehydrogenases from Acinetobacter sp. ADP1; glutamate dehydrogenases (EC 1.4.1.3) from Ralstonia, in particular glutamate dehydrogenases from Ralstonia solanacearum; glutamate dehydrogenases acting with NADPH as cofactor (EC 1.4.1.4) from Salmonella, in particular glutamate dehydrogenases from Salmonella typhimurium; glutamate dehydrogenases (EC 1.4.1.4) from Saccharomyces, in particular glutamate dehydrogenases from Saccharomyces cerevisiae; glutamate dehydrogenases (EC 1.4.1.4) from Brevibacterium, in particular glutamate dehydrogenases from Brevibacterium flavum; and leucine dehydrogenases from Bacillus, in particular leucine dehydrogenases from Bacillus cereus or Bacillus subtilis.

Another suitable amino acid dehydrogenase may be selected from the group of lysine 6-dehydrogenases from Agrobacterium tumefaciens or Geobacillus stearothermophilus; or from the group of leucine dehydrogenases from Bacillus cereus or Bacillus subtilis.

AKP, to be used to prepare 6-AAP, may in principle be obtained in any way. For instance, AKP may be obtained based on a method as described by H. Jäger et al. Chem. Ber. 1959, 92, 2492-2499. AKP can be prepared by alkylating cyclopentanone with diethyl oxalate using sodium ethoxide as a base, refluxing the resultant product in a strong acid (2 M HCl) and recovering the product, e.g. by crystallisation from toluene.

It is also possible to obtain AKP from a natural source, e.g. from methanogenic Archaea, from Asplenium septentrionale, or from Hydnocarpus anthelminthica. AKP may for instance be extracted from such organism, or a part thereof, e.g. from Hydnocarpus anthelminthica seeds. A suitable extraction method may e.g. be based on the method described in A. I. Virtanen and A. M. Berg in Acta Chemica Scandinavica 1954, 6, 1085-1086, wherein the extraction of amino acids and AKP from Asplenium, using 70% ethanol, is described.

In a specific embodiment, AKP is prepared in a method comprising converting alpha-ketoglutaric acid (AKG) into alpha-ketoadipic acid (AKA) and converting alpha-ketoadipic acid into alpha-ketopimelic acid. This reaction may be catalysed by a biocatalyst. AKG may, e.g., be prepared biocatalytically from a carbon source, such as a carbohydrate, in a manner known in the art per se.

A suitable biocatalyst for preparing AKP from AKG may in particular be selected amongst biocatalysts catalysing C₁-elongation of alpha-ketoglutaric acid into alpha-ketoadipic acid and/or C₁-elongation of alpha-ketoadipic acid into alpha-ketopimelic acid.

In a specific embodiment, the preparation of AKP is catalysed by a biocatalyst comprising
a. an AksA enzyme or an homologue thereof;
b. at least one enzyme selected from the group of AksD enzymes, AksE enzymes, homologues of AksD enzymes and homologues of AksE enzymes; and
c. an AksF enzyme or a homologue thereof.

Preferably, the catalyst comprises both an enzyme selected from the group of AksD enzymes and homologues thereof and an enzyme selected from the group of AksE enzymes and homologues thereof. Said AksD enzyme or its homologue and said AksE enzyme typically form a heterodimer.

One or more of the AksA, AksD, AksE, AksF enzymes or homologues thereof may be found in an organism selected from the group of methanogenic archaea, preferably selected from the group of Methanococcus, Methanocaldococcus, Methanosarcina, Methanothermobacter, Methanosphaera, Methanopyrus and Methanobrevibacter.

In a specific embodiment, the biocatalyst catalysing the preparation of AKP from alpha-ketoglutaric acid (AKG) comprises an enzyme system catalysing the conversion of alpha-ketoglutaric acid into alpha-ketoadipic acid, wherein said enzyme system forms part of the alpha-amino adipate pathway for lysine biosynthesis. The term ‘enzyme system’ is in particular used herein for a single enzyme or a group of enzymes whereby a specific conversion can be catalysed.

The preparation of AKP from AKG may comprise one or more biocatalytic reactions with known or unknown intermediates e.g. the conversion of AKG into AKA or the conversion of AKA into AKP. Such system may be present inside a cell or isolated from a cell. The enzyme system may in particular be from an organism selected from the group of yeasts, fungi, archaea and bacteria, in particular from the group of Penicillium, Cephalosporium, Paelicomyces, Trichophytum, Aspergillus, Phanerochaete, Emericella, Ustilago, Schizosaccharomyces, Saccharomyces, Candida, Yarrowia, Pichia, Kluyveromyces, Thermus, Deinococcus, Pyrococcus, Sulfolobus, Thermococcus, Methanococcus, Methanocaldococcus, Methanosphaera, Methanopyrus, Methanobrevibacter, Methanosarcina and Methanothermobacter.

In a specific embodiment, the biocatalyst catalysing the preparation of AKP from alpha-ketoglutaric acid comprises an enzyme system catalysing the conversion of alpha-ketoglutaric acid into alpha-ketoadipic acid, wherein at least one of the enzymes of the enzyme system originates from nitrogen fixing bacteria selected from the group of cyanobacteria, rhizobiales, γ-proteobacteria and actinobacteria, in particular from the group of Anabaena, Microcystis, Synechocystis, Rhizobium, Bradyrhizobium, Pseudomonas, Azotobacter, Klebsiella and Frankia.

Examples of homologues for these Aks enzymes and the genes encoding these enzymes are given in the Tables 1A and 1B on the following pages.

| | Enzyme | | | |
|---|---|---|---|---|
| Step | name | Organism | gene | Protein |
| 1 | AksA | Methanocaldococcus jannashii | MJ0503 | NP_247479 |
| | | Methanothermobacter | MTH1630 | NP_276742 |
| | | thermoautotropicum ΔH | | |
| | | Methanococcus maripaludis S2 | MMP0153 | NP_987273 |
| | | Methanococcus maripaludis C5 | MmarC5_1522 | YP_001098033 |
| | | Methanococcus maripaludis C7 | MmarC7_1153 | YP_001330370 |
| | | Methanospaera stadtmanae | Msp_0199 | YP_447259 |
| | | DSM 3091 | | |
| | | Methanopyrus kandleri AV19 | MK1209 | NP_614492 |
| | | Methanobrevibacter smithii | Msm_0722 | YP_001273295 |
| | | ATCC35061 | | |
| | | Methanococcus vannielii SB | Mevan_1158 | YP_001323668 |
| | | Klebsiella pneumoniae | nifV | P05345 |
| | | Azotobacter vinelandii | nifV | P05342 |
| | | Pseudomonas stutzerii | nifV | ABP79047 |
| | | Methanococcus aeolicus Nankai 3 | Maeo_0994 | YP_001325184 |
| 2, 3 | AksD | Methanocaldococcus jannashii | MJ1003 | NP_247997 |
| | | Methanothermobacter | MTH1386 | NP_276502 |
| | | thermoautotropicum ΔH | | |
| | | Methanococcus maripaludis S2 | Mmp1480 | NP_988600 |
| | | Methanococcus maripaludis C5 | MmarC5_0098 | YP_001096630 |
| | | Methanococcus maripaludis C7 | MmarC7_0724 | YP_001329942 |
| | | Methanospaera stadtmanae | Msp_1486 | YP_448499 |
| | | DSM 3091 | | |
| | | Methanopyrus kandleri AV19 | MK1440 | NP_614723 |
| | | Methanobrevibacter smithii | Msm_0723 | YP_001273296 |
| | | ATCC35061 | | |
| | | Methanococcus vannielii SB | Mevan_0789 | YP_001323307 |
| | | Methanococcus aeolicus Nankai 3 | Maeo_0311 | YP_001324511 |
| | | Methanosarcina acetivorans | MA3085* | NP_617978* |
| | | Methanospirillum hungatei JF-1 | Mhun_1800* | YP_503240* |
| | | Methanosaeta thermophila PT | Mthe_0788* | YP_843217* |
| | | Methanosphaera stadtmanae | Msp_1100* | YP_448126* |
| | | DSM 3091 | | |

References to gene and protein can be found via www.ncbi.nlm.nih.gov/ (for listed gene/protein marked with an *: as available on 2 Mar. 2010, for the others: as available on 15 Apr. 2008).

| | Enzyme | | | |
|---|---|---|---|---|
| Stp | name | Orgamism | gene | Protein |
| 2, 3 | AksE | Methanocaldococcus jannashii | MJ1271 | NP_248267 |
| | | Methanothermobacter | MTH1387 | NP_276503 |
| | | thermoautotropicum ΔH | | |
| | | Methanococcus maripaludis S2 | MMP0381 | NP_987501 |
| | | Methanococcus maripaludis C5 | MmarC5_1257 | YP_001097769 |
| | | Methanococcus maripaludis C7 | MmarC7_1379 | YP_001330593 |
| | | Methanospaera stadtmanae | Msp_1485 | YP_448498 |
| | | DSM 3091 | | |
| | | Methanopyrus kandleri AV19 | MK0781 | NP_614065 |
| | | Methanobrevibacter smithii | Msm_0847 | YP_001273420 |
| | | ATCC35061 | | |
| | | Methanococcus vannielii SB | Mevan_1368 | YP_001323877 |
| | | Methanococcus aeolicus Nankai 3 | Maeo_0652 | YP_001324848 |
| | | Methanosarcina acetivorans | MA3751* | NP_618624* |
| | | Methanospirillum hungatei JF-1 | Mhun_1799* | YP_503239* |
| | | Methanosphaera stadtmanae | Msp_0374* | YP_447420* |
| | | DSM 3091 | | |
| | | Methanosaeta thermophila PT | Mthe_0853* | YP_843282* |
| 4 | AksF | Methanocaldococcus jannashii | MJ1596 | NP_248605 |
| | | Methanothermobacter | MTH184 | NP_275327 |
| | | thermoautotropicum ΔH | | |
| | | Methanococcus maripaludis S2 | MMP0880 | NP988000 |
| | | Methanococcus maripaludis C5 | MmarC5_0688 | YP001097214 |
| | | Methanococcus maripaludis C7 | MmarC7_0128 | YP_001329349 |
| | | Methanospaera stadtmanae | Msp_0674 | YP_447715 |
| | | DSM 3091 | | |
| | | Methanopyrus kandleri AV19 | MK0782 | NP_614066 |
| | | Methanobrevibacter smithii | Msm_0373 | YP001272946 |
| | | ATCC35061 | | |
| | | Methanococcus vannielii SB | Mevan_0040 | YP_001322567 |
| | | Methanococcus aeolicus Nankai 3 | Maeo_1484 | YP_001325672 |
| | | Methanosarcina acetivorans | MA3748* | NP_618621* |
| | | Methanospirillum hungatei JF-1 | Mhun_1797* | YP_503237* |
| | | Methanosphaera stadtmanae | Msp_0674* | YP_447715* |
| | | DSM 3091 | | |
| | | Methanosaeta thermophila PT | Mthe_0855* | YP_843284* |
| | | Methanobrevibacter smithii | Msm_1298* | YP_001273871* |
| | | ATCC 35061 | | |

References to gene and protein can be found via www.ncbi.nlm.nih.gov/ ((for listed gene/protein marked with an *: as available on 2 Mar. 2010, for the others:as available on 15 Apr. 2008).

The 6-ACA obtained in a method according to the invention can be isolated from the biocatalyst, as desired. A suitable isolation method can be based on methodology commonly known in the art.

If desired, 6-ACA obtained in accordance with the invention can be cyclised to form caprolactam, e.g. as described in U.S. Pat. No. 6,194,572.

Reaction conditions for any biocatalytic step in the context of the present invention may be chosen depending upon known conditions for the biocatalyst, in particular the enzyme, the information disclosed herein and optionally some routine experimentation.

In principle, the pH of the reaction medium used may be chosen within wide limits, as long as the biocatalyst is active under the pH conditions. Alkaline, neutral or acidic conditions may be used, depending on the biocatalyst and other factors. In case the method includes the use of a micro-organism, e.g. for expressing an enzyme catalysing a method of the invention, the pH is selected such that the micro-organism is capable of performing its intended function or functions. The pH may in particular be chosen within the range of four pH units below neutral pH and two pH units above neutral pH, i.e. between pH 3 and pH 9 in case of an essentially aqueous system at 25° C. A system is considered aqueous if water is the only solvent or the predominant solvent (>50 wt. %, in particular >90 wt. %, based on total liquids), wherein e.g. a minor amount of alcohol or another solvent (<50 wt. %, in particular <10 wt. %, based on total liquids) may be dissolved (e.g. as a carbon source) in such a concentration that micro-organisms which may be present remain active. In particular in case a yeast and/or a fungus is used, acidic conditions may be preferred, in particular the pH may be in the range of pH 3 to pH 8, based on an essentially aqueous system at 25° C. If desired, the pH may be adjusted using an acid and/or a base or buffered with a suitable combination of an acid and a base.

In principle, the incubation conditions can be chosen within wide limits as long as the biocatalyst shows sufficient activity and/or growth. This includes aerobic, micro-aerobic, oxygen limited and anaerobic conditions.

Anaerobic conditions are herein defined as conditions without any oxygen or in which substantially no oxygen is consumed by the biocatalyst, in particular a micro-organism, and usually corresponds to an oxygen consumption of less than 5 mmol/l.h, in particular to an oxygen consumption of less than 2.5 mmol/l.h, or less than 1 mmol/l.h.

Aerobic conditions are conditions in which a sufficient level of oxygen for unrestricted growth is dissolved in the medium, able to support a rate of oxygen consumption of at least 10 mmol/l.h, more preferably more than 20 mmol/l.h, even more preferably more than 50 mmol/l.h, and most preferably more than 100 mmol/l.h.

Oxygen-limited conditions are defined as conditions in which the oxygen consumption is limited by the oxygen transfer from the gas to the liquid. The lower limit for oxygen-limited conditions is determined by the upper limit for anaerobic conditions, i.e. usually at least 1 mmol/l.h, and in particular at least 2.5 mmol/l.h, or at least 5 mmol/l.h. The upper limit for oxygen-limited conditions is determined by the lower limit for aerobic conditions, i.e. less than 100 mmol/l.h, less than 50 mmol/l.h, less than 20 mmol/l.h, or less than to 10 mmol/l.h.

Whether conditions are aerobic, anaerobic or oxygen limited is dependent on the conditions under which the method is carried out, in particular by the amount and composition of ingoing gas flow, the actual mixing/mass transfer properties of the equipment used, the type of micro-organism used and the micro-organism density.

In principle, the temperature used is not critical, as long as the biocatalyst, in particular the enzyme, shows substantial activity. Generally, the temperature may be at least 0° C., in particular at least 15° C., more in particular at least 20° C. A desired maximum temperature depends upon the biocatalyst. In general such maximum temperature is known in the art, e.g. indicated in a product data sheet in case of a commercially available biocatalyst, or can be determined routinely based on common general knowledge and the information disclosed herein. The temperature is usually 90° C. or less, preferably 70° C. or less, in particular 50° C. or less, more in particular or 40° C. or less.

In particular if a biocatalytic reaction is performed outside a host organism, a reaction medium comprising an organic solvent may be used in a high concentration (e.g. more than 50%, or more than 90 wt. %), in case an enzyme is used that retains sufficient activity in such a medium.

In an advantageous method 6-ACA is prepared making use of a whole cell biotransformation of the substrate for 6-ACA or an intermediate for forming 6-ACA (such as AKP or AAP), said method comprising the use of a micro-organism in which one or more biocatalysts (usually one or more enzymes) catalysing the biotransformation are produced, such as one or more biocatalysts selected from the group of biocatalysts capable of catalysing the conversion of AKP to AAP and biocatalysts capable of catalysing the conversion of AAP to 6-ACA. In a preferred embodiment the micro-organism is capable of producing a decarboxylase and/or at least one enzyme selected from amino acid dehydrogenases and aminotransferases are produced. capable of catalysing a reaction step as described above, and a carbon source for the micro-organism.

The carbon source may in particular contain at least one compound selected from the group of monohydric alcohols, polyhydric alcohols, carboxylic acids, carbon dioxide, fatty acids, glycerides, including mixtures comprising any of said compounds. Suitable monohydric alcohols include methanol and ethanol, Suitable polyols include glycerol and carbohydrates. Suitable fatty acids or glycerides may in particular be provided in the form of an edible oil, preferably of plant origin.

In particular a carbohydrate may be used, because usually carbohydrates can be obtained in large amounts from a biologically renewable source, such as an agricultural product, preferably an agricultural waste-material. Preferably a carbohydrate is used selected from the group of glucose, fructose, sucrose, lactose, saccharose, starch, cellulose and hemi-cellulose. Particularly preferred are glucose, oligosaccharides comprising glucose and polysaccharides comprising glucose.

A cell, in particular a recombinant cell, comprising one or more biocatalysts (usually one or more enzymes) for catalysing a reaction step in a method of the invention can be constructed using molecular biological techniques, which are known in the art per se. For instance, if one or more biocatalysts are to be produced in a recombinant cell (which may be a heterologous system), such techniques can be used to provide a vector (such as a recombinant vector) which comprises one or more genes encoding one or more of said biocatalysts. One or more vectors may be used, each comprising one or more of such genes. Such vector can comprise one or more regulatory elements, e.g. one or more promoters, which may be operably linked to a gene encoding an biocatalyst.

As used herein, the term “operably linked” refers to a linkage of polynucleotide elements (or coding sequences or nucleic acid sequence) in a functional relationship. A nucleic acid sequence is “operably linked” when it is placed into a functional relationship with another nucleic acid sequence. For instance, a promoter or enhancer is operably linked to a coding sequence if it affects the transcription of the coding sequence.

As used herein, the term “promoter” refers to a nucleic acid fragment that functions to control the transcription of one or more genes, located upstream with respect to the direction of transcription of the transcription initiation site of the gene, and is structurally identified by the presence of a binding site for DNA-dependent RNA polymerase, transcription initiation sites and any other DNA sequences, including, but not limited to transcription factor binding sites, repressor and activator protein binding sites, and any other sequences of nucleotides known to one of skilled in the art to act directly or indirectly to regulate the amount of transcription from the promoter. A “constitutive” promoter is a promoter that is active under most environmental and developmental conditions. An “inducible” promoter is a promoter that is active under environmental or developmental regulation. The term “homologous” when used to indicate the relation between a given (recombinant) nucleic acid or polypeptide molecule and a given host organism or host cell, is understood to mean that in nature the nucleic acid or polypeptide molecule is produced by a host cell or organisms of the same species, preferably of the same variety or strain.

The promoter that could be used to achieve the expression of the nucleic acid sequences coding for an enzyme for use in a method of the invention, in particular an aminotransferase, an amino acid dehydrogenase or a decarboxylase, such as described herein above may be native to the nucleic acid sequence coding for the enzyme to be expressed, or may be heterologous to the nucleic acid sequence (coding sequence) to which it is operably linked. Preferably, the promoter is homologous, i.e. endogenous to the host cell.

If a heterologous promoter (to the nucleic acid sequence encoding for the enzyme of interest) is used, the heterologous promoter is preferably capable of producing a higher steady state level of the transcript comprising the coding sequence (or is capable of producing more transcript molecules, i.e. mRNA molecules, per unit of time) than is the promoter that is native to the coding sequence. Suitable promoters in this context include both constitutive and inducible natural promoters as well as engineered promoters, which are well known to the person skilled in the art.

A “strong constitutive promoter” is one which causes mRNAs to be initiated at high frequency compared to a native host cell. Examples of such strong constitutive promoters in Gram-positive micro-organisms include SP01-26, SP01-15, veg, pyc (pyruvate carboxylase promoter), and amyE.

Examples of inducible promoters in Gram-positive micro-organisms include, the IPTG inducible Pspac promoter, the xylose inducible PxylA promoter.

Examples of constitutive and inducible promoters in Gram-negative microorganisms include, but are not limited to, tac, tet, trp-tet, Ipp, lac, lpp-lac, lacIq, T7, T5, T3, gal, trc, ara (P_{BAD}), SP6, λ-P_{R}, and λ-P_{L}.

Promoters for (filamentous) fungal cells are known in the art and can be, for example, the glucose-6-phosphate dehydrogenase gpdA promoters, protease promoters such as pepA, pepB, pepC, the glucoamylase glaA promoters, amylase amyA, amyB promoters, the catalase catR or catA promoters, glucose oxidase goxC promoter, beta-galactosidase lacA promoter, alpha-glucosidase aglA promoter, translation elongation factor tefA promoter, xylanase promoters such as xlnA, xlnB, xlnC, xlnD, cellulase promoters such as eglA, eglB, cbhA, promoters of transcriptional regulators such as areA, creA, xlnR, pacC, prtT, or another promotor, and can be found among others at the NCBI website (http://www.ncbi.nlm.nih.gov/entrez/).

The term “heterologous” when used with respect to a nucleic acid (DNA or RNA) or protein refers to a nucleic acid or protein that does not occur naturally as part of the organism, cell, genome or DNA or RNA sequence in which it is present, or that is found in a cell or location or locations in the genome or DNA or RNA sequence that differ from that in which it is found in nature. Heterologous nucleic acids or proteins are not endogenous to the cell into which it is introduced, but has been obtained from another cell or synthetically or recombinantly produced. Generally, though not necessarily, such nucleic acids encode proteins that are not normally produced by the cell in which the DNA is transcribed or expressed. Similarly exogenous RNA encodes for proteins not normally expressed in the cell in which the exogenous RNA is present. Heterologous nucleic acids and proteins may also be referred to as foreign nucleic acids or proteins. Any nucleic acid or protein that one of skill in the art would recognize as heterologous or foreign to the cell in which it is expressed is herein encompassed by the term heterologous nucleic acid or protein.

A method according to the invention may be carried out in a host organism, which may be novel. The host organism relates to a recombinant cell comprising a gene encoding a heterologous enzyme.

Accordingly, the invention also relates to a recombinant host cell comprising a gene encoding a heterologous enzyme having alpha-aminopimelic acid decarboxylase activity, wherein said enzyme comprises an amino acid sequence represented by any of the SEQUENCE ID NO's: 2, 5, 8 and 11 and homologues of said sequences. The gene may form part of one or more vectors.

The invention also relates to a novel vector comprising one or more genes encoding an enzyme having alpha-aminopimelic acid decarboxylase activity and comprising an amino acid sequence represented by any of the SEQUENCE ID NO's: 2, 5, 8 and 11 and homologues of said sequences

The nucleic acid sequence may in particular be a wild type sequence that is heterologous to the host cell (i.e. found naturally in a different organism) or a codon optimised sequence. Suitable sequences include any of the SEQUENCE ID NO's: 1, 3, 4, 6, 7, 9 and 10 and functional analogues thereof. Preferred sequences include sequence according to any of the SEQUENCE ID NO's: 3, 6, and 9 and functional analogues thereof having a similar, the same or a better level of expression in an Escherichia host cell (in particular E. coli) or another host cell of interest.

In a specific embodiment, the host cell according to the invention is a host cell further comprising a nucleic acid sequence encoding a biocatalyst capable of catalysing a transamination reaction or a reductive amination reaction to form alpha-aminopimelic acid from alpha-ketopimelic acid. Said sequence may be part of a vector or may have been inserted into the chromosomal DNA.

In a preferred embodiment, the host cell comprises a nucleic acid sequence encoding an enzyme, capable of catalysing the conversion of AKP to AAP, according to Sequence ID No.: 14, 16, 20, 22, 24, 25, 27, 29, 31, 33, 35, 37, 39, or a functional analogue thereof, which may be a wild type or non-wild type sequence.

In a specific embodiment, the host cell comprises one or more enzymes catalysing the formation of AKP from AKG (see also above). Use may be made of an enzyme system forming part of the alpha-amino adipate pathway for lysine biosynthesis. The term ‘enzyme system’ is in particular used herein for a single enzyme or a group of enzymes whereby a specific conversion can be catalysed. Said conversion may comprise one or more chemical reactions with known or unknown intermediates e.g. the conversion of AKG into AKA or the conversion of AKA into AKP. Such system may be present inside a cell or isolated from a cell. It is known that aminotransferases often have a wide substrate range. If present, it may be desired to decrease activity of one or more such enzymes in a host cell such that activity in the conversion of AKA to alpha-aminoadipate (AAA) is reduced, whilst maintaining relevant catalytic functions for biosynthesis of other amino acids or cellular components. Also a host cell devoid of any other enzymatic activity resulting in the conversion of AKA to an undesired side product is preferred.

In a preferred host cell, suitable for preparing AAP making use of a whole cell biotransformation process, one or more biocatalysts capable of catalysing at least one reaction step in the preparation of alpha-ketopimelic acid from alpha-ketoglutaric acid are encoded for. Suitable biocatalysts are, e.g., as described above when discussing the preparation of AKP.

The host cell may for instance be selected from bacteria, yeasts or fungi. In particular the host cell may be selected from the genera selected from the group of Aspergillus, Penicillium, Saccharomyces, Kluyveromyces, Pichia, Candida, Hansenula, Bacillus, Corynebacterium, Pseudomonas, Gluconobacter, Methanococcus, Methanobacterium, Methanocaldococcus and Methanosarcina and Escherichia. Herein, usually one or more encoding nucleic acid sequences as mentioned above have been cloned and expressed.

In particular, the host strain and, thus, a host cell suitable for the biochemical synthesis of 6-ACA may be selected from the group of Escherichia coli, Bacillus subtilis, Bacillus amyloliquefaciens, Corynebacterium glutamicum, Aspergillus niger, Penicillium chrysogenum, Saccharomyces cervisiae, Hansenula polymorpha, Candida albicans, Kluyveromyces lactis, Pichia stipitis, Pichia pastoris, Methanobacterium thermoautothrophicum ΔH, Methanococcus maripaludis, Methanococcus voltae, Methanosarcina acetivorans, Methanosarcina barkeri and Methanosarcina mazei host cells. In a preferred embodiment, the host cell is capable of producing lysine (as a precursor).

The host cell may be in principle a naturally occurring organism or may be an engineered organism. Such an organism can be engineered using a mutation screening or metabolic engineering strategies known in the art. In a specific embodiment, the host cell naturally comprises (or is capable of producing) one or more of the enzymes suitable for catalysing a reaction step in a method of the invention, such as one or more activities selected from the group of decarboxylases, aminotransferases and amino acid dehydrogenases capable of catalysing a reaction step in a method of the invention. For instance E. coli may naturally be capable of producing an enzyme catalysing a transamination in a method of the invention. It is also possible to provide a recombinant host cell with both a recombinant gene encoding an aminotransferase or amino acid dehydrogenase capable of catalysing a reaction step in a method of the invention and a recombinant gene encoding a decarboxylase gene capable of catalysing a reaction step in a method of the invention.

For instance a host cell may be selected of the genus Corynebacterium, in particular C. glutamicum, enteric bacteria, in particular Escherichia coli, Bacillus, in particular B. subtilis and B. methanolicus, and Saccharomyces, in particular S. cerevisiae. Particularly suitable are C. glutamicum or B. methanolicus strains which have been developed for the industrial production of lysine.

For such method in particular a biocatalyst may be used having aminotransferase activity or reductive amination activity as described above.

Further, the invention is directed to a novel polynucleotide encoding for an enzyme that may be used in accordance with the invention. Accordingly, the invention is further directed to a polynucleotide comprising a sequence according to any of the SEQUENCE ID NO's: 3, 6, and 9 and functional analogues thereof having a similar, the same or a better level of expression in an Escherichia host cell. To the best of the inventors' knowledge these polynucleotides do not occur in nature. In particular, in as far as they would occur in nature, any of these polynucleotides is in particular claimed isolated from any organism in which it naturally occurs.

Next, the invention will be illustrated by the following examples.

### EXAMPLES

### General Methods

### Molecular and Genetic Techniques

Standard genetic and molecular biology techniques are generally known in the art and have been previously described (Maniatis et al. 1982 “Molecular cloning: a laboratory manual”. Cold Spring Harbor Laboratory, Cold Spring Harbor, N.Y.; Miller 1972 “Experiments in molecular genetics”, Cold Spring Harbor Laboratory, Cold Spring Harbor; Sambrook and Russell 2001 “Molecular cloning: a laboratory manual” (3rd edition), Cold Spring Harbor Laboratory, Cold Spring Harbor Laboratory Press; F. Ausubel et al, eds., “Current protocols in molecular biology”, Green Publishing and Wiley Interscience, New York 1987).

### Plasmids and Strains

pBAD/Myc-His C was obtained from Invitrogen (Carlsbad, Calif., USA). Plasmid pBAD/Myc-His-DEST constructed as described in WO2005/068643, was used for protein expression. E. coli TOP10 (Invitrogen, Carlsbad, Calif., USA) was used for all cloning procedures and for expression of target genes in the pBAD-system. E. coli BL21(DE3) and pET-26b(+) were obtained from Novagen (EMD/Merck, Nottingham, UK).

### Media

2XTY medium (16 g/l tryptone, 10 g/l yeast extract, 5 g/l NaCl) was used for growth of E. coli. Antibiotics (100 μg/ml carbenicillin, 50 μg/ml neomycin) were supplemented to maintain plasmids. For induction of gene expression under control of the P_{BAD} promoter in pBAD/Myc-HisC derived plasmids, L-arabinose was added to a final concentration of 0.02% (w/v). For induction of gene expression under control of the T7-promoter in pET-26b(+) derived plasmids, IPTG was added to a final concentration of 1 mM.

### Identification of Plasmids

Plasmids carrying the different genes were identified by genetic, biochemical, and/or phenotypic means generally known in the art, such as resistance of transformants to antibiotics, PCR diagnostic analysis of transformant or purification of plasmid DNA, restriction analysis of the purified plasmid DNA or DNA sequence analysis.

### HPLC-MS Analysis for the Determination of 6-ACA

### Calibration:

The calibration was performed by an external calibration line of G-ACA (m/z 132→m/z 114, Rt 7.5 min). All the LC-MS experiments were performed on an Agilent 1100, equipped with a quaternary pump, degasser, autosampler, column oven, and a single-quadrupole MS (Agilent, Waldbronn, Germany). The LC-MS conditions were:

Column: 50*4 Nucleosil (Mancherey-Nagel)+250×4.6 Prevail C18 (Alltech), both at room temperature (RT)
Eluent: A=0.1 (v/v) formic acid in ultrapure water
B=Acetonitrile (pa, Merck)
   Flow: 1.0 ml/min, before entering the MS the flow was split 1:3
   Gradient: The gradient was started at t=0 minutes with 100% (v/v) A, remaining for 15 minutes and changed within 15 minutes to 80% (v/v) B (t=30 minutes). From 30 to 31 minutes the gradient was kept at constant at 80% (v/v) B.
   Injection volume: 5 μl
   MS detection: ESI(+)-MS

The electrospray ionization (ESI) was run in the positive scan mode with the following conditions; m/z 50-500, 50 V fragmentor, 0.1 m/z step size, 350° C. drying gas temperature, 10 L N₂/min drying gas, 50 psig nebuliser pressure and 2.5 kV capillary voltage.

### Cloning of Target Genes

### Design of Expression Constructs

For the cloning of target genes by homologous recombination in pBAD-DEST plasmids attB sites were added to all genes upstream of the ribosomal binding site and start codon and downstream of the stop codon to facilitate cloning using the Gateway technology (Invitrogen, Carlsbad, Calif., USA).

### Example 1

### Conversion of AKP to AAP

This Example is taken from the Examples of WO 2009/113855 which are incorporated herein by reference, in particular the parts describing the construction of the cells.

A reaction mixture was prepared comprising 10 mM alpha-ketopimelic acid, 20 mM L-alanine, and 50 μM pyridoxal 5′-phosphate in 50 mM potassium phosphate buffer, pH 7.0. 800 μl of the reaction mixture were dispensed into each well of the well plates. To start the reaction, 200 μl of the cell lysates were added, to each of the wells. Reaction mixtures were incubated on a shaker at 37° C. for 24 h. Furthermore, a chemical blank mixture (without cell free extract) and a biological blank (E. coli TOP10 with pBAD/Myc-His C) were incubated under the same conditions. Samples were analysed by HPLC-MS. The results are summarised in the following table.

TABLE 1
AAP formation from AKP in the presence of aminotransferases

| | AAP concentration [mg/kg] |
|---|---|
| Biocatalyst | (after 24 hrs) |
| E. coli TOP10/pBAD-Vfl_AT | 3.7 |
| E. coli TOP10/pBAD-Psy_AT | 15.8 |
| E. coli TOP10/pBAD-Bsu_gi16078032_AT | 11.2 |
| E. coli TOP10/pBAD-Rsp_AT | 9.8 |
| E. coli TOP10/pBAD-Bsu_gi16080075_AT | 4.6 |
| E. coli TOP10/pBAD-Lpn_AT | 5.4 |
| E. coli TOP10/pBAD-Neu_AT | 7.7 |
| E. coli TOP10/pBAD-Ngo_AT | 5.1 |
| E. coli TOP10/pBAD-Pae_gi9951299_AT | 5.6 |
| E. coli TOP10/pBAD-Rpa_AT | 5.4 |
| E. coli TOP10 with pBAD/Myc-His C | 1.4 |
| (biological blank) | |
| None (chemical blank) | 0 |

It is shown that the formation of AAP from AKP is catalysed by the biocatalyst.

### Example 2

### Biocatalytic Preparation of 6-ACA from AAP

### Gene Synthesis and Construction of Plasmids

Synthetic genes were obtained from DNA2.0 and codon optimised for expression in E. coli according to standard procedures of DNA2.0. The diaminopimelate decarboxylase genes from Thermotoga maritima [SEQ ID No. 1], Corynebacterium glutamicum [SEQ ID No. 4], and Bacillus subtilis [SEQ ID No. 7] encoding the amino acid sequences of the T. maritima diaminopimelate decarboxylase Q9X1K5 [SEQ ID No. 2], C. glutamicum diaminopimelate decarboxylase P09890 [SEQ ID No. 5], and B. subtilis diaminopimelate decarboxylase P23630 [SEQ ID No. 8], respectively, were codon optimised and the resulting sequences [SEQ ID No. 3], [SEQ ID No. 6] and [SEQ ID No. 9] were obtained by DNA synthesis. The gene constructs were cloned into pBAD/Myc-His-DEST expression vectors using the Gateway technology (Invitrogen) via the introduced attB sites and pDONR201 (Invitrogen) as entry vector as described in the manufacturer's protocols (www.invitrogen.com). The gene constructs were cloned into pBAD/Myc-His-DEST expression vectors using the Gateway technology (Invitrogen) via the introduced attB sites and pDONR201 (Invitrogen) as entry vector as described in the manufacturer's protocols (www.invitrogen.com). This way the expression vectors pBAD-Tma_AAP-DC, pBAD-Cgl_AAP-DC, and pBAD-Bsu_AAP-DC were obtained, respectively. The corresponding expression strains were obtained by transformation of chemically competent E. coli TOP10 (Invitrogen) with the respective pBAD-expression vectors.

### Cloning by PCR

The diaminopimelate decarboxylase gene from Pseudomonas putida DSM 50026 [SEQ ID No. 10] coding for P. putida diaminopimelate decarboxylase [SEQ ID No. 11] was amplified from genomic DNA of P. putida DSM 50026 by PCR. Genomic DNA of P. putida DSM 50026 was isolated following the general protocol of the QIAGEN Genomic DNA Handbook (QIAGEN, Hilden, Germany) for the isolation of chromosomal DNA from gram negative bacteria. The raw preparation was purified by using a QIAGEN Genomic-tip 500/G column (QIAGEN, Hilden, Germany) according to the manufacturer's procedure. PCR Supermix High Fidelity (Invitrogen) was used according to the manufacturer's specifications with the following oligonucleotides:

Forward primer [SEQ ID No. 12]:
5′-gccatatgaa cgctttcaac taccgcga-3′
Reverse primer [SEQ ID No. 13]:
5′-gcaagcttac tccggcagca ggctttcgc-3′

PCR reactions were analysed by agarose gel electrophoresis and PCR products of the correct size were eluted from the gel using the QIAquick PCR purification kit (Qiagen, Hilden, Germany) and digested with NdeI and HindIII. The digested PCR products were gel purified and ligated into pET26b(+) which had been opened with the NdeI and HindIII using T4 DNA ligase (Invitrogen) according to the manufacturer's specifications. This way the expression vector pET26-DC-Pp1/8 was obtained. The gene sequence was verified by DNA sequencing. The corresponding expression strain was obtained by transformation of chemically competent E. coli BL21(DE3) (Invitrogen) with pET26-DC-Pp1/8.

### Growth of E. Coli for Protein Expression and Cell-Free Extract Preparation

5 ml 2XTY precultures of containing 50 μg/ml antibiotic were inoculated and cultivated over night at 28° C. and 180 rpm on an orbitary shaker. From these precultures expression cultures were inoculated in Erlenmeyer flasks containing 50-100 ml 2*TY plus 50 μg/ml antibiotic to a start cell density of OD₆₂₀=0.05. These cultures were incubated at 28° C. and 180 rpm on an orbitary shaker. In the middle of the exponential growth phase (OD₆₂₀ of about 0.6) the expression of the target genes was induced by the addition of 0.02% (w/v) L-arabinose or 1 mM IPTG to the culture flasks. After induction the cultivation was continued at 28° C. and 180 rpm on an orbitary shaker over night (about 20 h). Subsequently the cells were harvested by centrifugation at 5,000×g for 10 min at 4° C. The supernatant was discarded and the cells were resuspended and weighed. The cell pellets were resuspended in twice the volume of wet weight of ice-cold 50 mM KPᵢ buffer pH 7.5 containing 0.1 mM PLP. Cell-free extracts (CFEs) were obtained by sonification of the cell suspensions using a Sonics (Meyrin/Satigny, Switzerland) Vibra-Cell VCX130 sonifier (output 100%, 10 s on/10 s off, for 10 min) with cooling in an ice/acetone bath and centrifugation in an Eppendorf (Hamburg, Germany) 5415R centrifuge at 13,000×g and 4° C. for 30 min. The supernatants (=CFEs) were transferred to fresh tubes and stored on ice for immediate use or stored at −20° C.

Biocatalytic Production of 6-ACA from AAP

In a total volume of 0.25 ml 0.1 ml of CFEs comprising overexpressed diaminopimelate decarboxylases from T. maritima [SEQ ID No. 2], C. glutamicum [SEQ ID No. 5], B. subtilis [SEQ ID No. 8], and P. putida [SEQ ID No. 11], respectively, were incubated with 50 mM α-aminopimelic acid (AAP) in the presence of 0.1 M potassium phosphate buffer pH 7.5 containing 0.1 mM PLP at 28° C. and shaking at 560 rpm. Reactions were stopped after 20 h and 40 h of incubation time by addition of 0.75 ml of a 1:1 acetonitrile/water mixture and centrifugation (20 min at 5000×g). As negative controls only buffer or a CFE comprising an overexpressed glucose dehydrogenase (GDH) from B. subtilis was incubated like the CFEs comprising overexpressed diaminopimelate decarboxylases. The reactions were analysed by HPLC-MS as described in the general methods. The results are given in Table 2.

TABLE 2
Decarboxylation of AAP to 6-ACA by recombinant
diaminopimelate decarboxylases

| | 6-ACA | 6-ACA |
|---|---|---|
| | after 20 h | after 40 h |
| Biocatalyst (E. coli cell free extracts) | [mmol/l] | [mmol/l] |
| T. maritima diaminopimelate decarboxylase | 1.23 | 2.05 |
| C. glutamicum diaminopimelate decarboxylase | 1.29 | 2.26 |
| B. subtilis diaminopimelate decarboxylase | 0.35 | 0.57 |
| P. putida diaminopimelate decarboxylase | 0.04 | 0.05 |
| buffer only (chemical blank) | <0.01 | <0.01 |
| glucose dehydrogenase (biological blank) | <0.01 | <0.01 |

The results show that the diaminopimelate decarboxylases from T. maritima [SEQ ID No. 2], C. glutamicum [SEQ ID No. 5], B. subtilis [SEQ ID No. 8], and P. putida [SEQ ID No. 11] are capable of also decarboxylating AAP to 6-ACA and are therefore also suitable AAP decarboxylases. This is surprising given the considerable structural differences between α-aminopimelic acid and diaminopimelic acid.

In parallel, the substrate specificity of the AAP decarboxylases from T. maritima [SEQ ID No. 2], C. glutamicum [SEQ ID No. 5], B. subtilis [SEQ ID No. 8], and P. putida [SEQ ID No. 11] was investigated by incubating the respective CFEs as described above in the presence of 50 mM α-aminoadipate and α-aminoglutarate (glutamate). The same analysis method as described above was used with the reference compounds α-aminoadipate, 5-aminovaleric acid, α-aminoglutarate, and 4-aminobuturic acid (Syncom, Groningen, The Netherlands). As negative controls only buffer or a CFE comprising an overexpressed glucose dehydrogenase from B. subtilis was incubated like the CFEs comprising overexpressed diaminopimelate decarboxylases. In none of the reaction mixtures the decarboxylation of α-aminoadipate to 5-aminovaleric acid or α-aminoglutarate to 4-aminobuturic acid, respectively, was detected. This shows that the AAP decarboxylases from T. maritima, C. glutamicum, B. subtilis, and P. putida are highly selective biocatalysts for the decarboxylation of AAP to 6-ACA compared to its shorter analogues α-aminoadipate and α-aminoglutarate.

### Example 3

### Chemical Conversion of AAP to Caprolactam

To a suspension of 1.5 grams of AAP in 21 ml cyclohexanone, 0.5 ml of cyclohexenone was added. The mixture was heated on an oil bath for 20 h at reflux (approximately 160° C.). After cooling to room temperature the reaction mixture was decanted and the clear solution was evaporated under reduced pressure. The remaining 2 grams of brownish oil were analyzed by ¹H-NMR and HPLC and contained 0.8 wt % caprolactam and 6 wt % of cyclic oligomers of caprolactam.

### Example 4

### Production of 6-ACA Using Diaminopimelate Decarboxylases In Vivo

### Protein Expression and Metabolite Production in E. Coli

Expression vectors pBAD-Tma_AAP-DC, pBAD-Cgl_AAP-DC, and pBAD-Bsu_AAP-DC (for a description see example 2.) encoding the amino acid sequences of the T. maritima diaminopimelate decarboxylase [SEQ ID No. 1], C. glutamicum [SEQ ID No. 4], and B. subtilis [SEQ ID No. 7], respectively were together with the empty pBAD vector transformed into E. coli strain BL-21(A1). Starter cultures were grown overnight in tubes with 10 ml 2XTY medium. 200 μl culture was transferred to shake flasks with 20 ml 2XTY medium. Flasks were incubated in an orbital shaker at 30° C. and 280 rpm. After 4 h IPTG was added at a final concentration of 0.1 mM and flasks were incubated for 4 h at 30° C. and 280 rpm. Cells from 20 ml culture were collected by centrifugation and resuspended in 4 ml 2XTY medium with 1% glycerol and 500 mg/l AKP in 24 well plates. After incubation for 48 h at 30° C. and 210 rpm cells were collected by centrifugation and pellet and supernatant were separated and stored at −20 C for analysis.

### UPLC-MS/MS Analysis Method for the Determination of 6-ACA and AAP

A Waters HSS T3 column 1.8 μm, 100 mm×2.1 mm was used for the separation of 6-ACA and AAP with gradient elution as depicted in Table 3. Eluent A consists of LC/MS grade water, containing 0.1% formic acid, and eluent B consists of acetonitrile, containing 0.1% formic acid. The flow-rate was 0.25 ml/min and the column temperature was kept constant at 40° C.

TABLE 3
gradient elution program used for the separation of 6-ACA and AAP

Time (min)

| | 0 | 5.0 | 5.5 | 10 | 10.5 | 15 |
|---|---|---|---|---|---|---|
| % A | 100 | 85 | 20 | 20 | 100 | 100 |
| % B | 0 | 15 | 80 | 80 | 0 | 0 |

A Waters micromass Quattro micro API was used in electrospray either positive or negative ionization mode, depending on the compounds to be analyzed, using multiple reaction monitoring (MRM). The ion source temperature was kept at 130° C., whereas the desolvation temperature is 350° C., at a flow-rate of 500 L/h r.

For 6-ACA and AAP the protonated molecule was fragmented with 13 eV, resulting in specific fragments from losses of H₂O, NH₃ and CO.

To determine concentrations, a calibration curve of external standards of synthetically prepared compounds was run to calculate a response factor for the respective ions. This was used to calculate the concentrations in samples. Samples were diluted appropriately (2-10 fold) in eluent A to overcome ion suppression and matrix effects.

### Analysis of Supernatant

Supernatant were diluted 5 times with water prior to UPLC-MS/MS analysis. Results (Table 4) clearly show presence of AAP in all strains analysed and it is contemplated that the conversion of AKP to AAP is catalyzed by a natural aminotransferase present in E. coli. Results also clearly show the presence of 6-ACA in recombinant strains while this can not be detected in the non-transformed BL21-A1 strain.

TABLE 4
production of AAP and 6-ACA in E. coli

| | Origin | | Mg/l |
|---|---|---|---|
| | diaminopimelate | mg/l 6-ACA | AAP after |
| plasmid | decarboxylase | after 48 hours | 48 hours |
| — | | 0 | 4.5 |
| pBAD-Tma_AAP-DC | T. maritima | 0.3 | 4.2 |
| pBAD-Cgl_AAP-DC | C. glutamicum | 0.05 | 5.1 |
| pBAD-Bsu_AAP-DC | B. subtilis | 0.15 | 4.7 |

### Example 5

### Homology Between Four Homologues Having AAP Decarboxylase Activity

### Method

The homology was performed using EMBOSS/needle which uses the Needleman-Wunsch alignment algorithm.

For all the comparisons the default settings were used:
# Matrix: EBLOSUM62
# Gap_penalty: 10.0
# Extend_penalty: 0.5

### Results of the Pairwise Comparison

A pair wise comparison of all 4 sequences has been performed. The %-ages of homology are shown in Tables 5 and 6.

TABLE 5
Homology %-age Identity

| | SEQID- | | |
|---|---|---|---|
| | No11 | SEQID-No8 | SEQID-No5 |
| SEQID-No2 | 38 | 33 | 30 |
| SEQID-No5 | 33 | 41 | |
| SEQID-No8 | 35 | | |

TABLE 6
Homology %-age Similarity

| | SEQID- | | |
|---|---|---|---|
| | No11 | SEQID-No8 | SEQID-No5 |
| SEQID-No2 | 56 | 52 | 45 |
| SEQID-No5 | 48 | 58 | |
| SEQID-No8 | 53 | | |

### Example 6

### Identification Of Enzymes Involved in the Conversion of AKP to AAP

### Growth of E. Coli for Protein Expression

Genes encoding enzymes having catalytic activity with respect to the conversion of AKP to AAP were identified by testing putative enzymes for said activity. Small scale growth of E. coli strains mutated in these genes (i.e. which genes were deleted) as identified in the E. coli KEIO mutant library (Baba T, Ara T, et al. (2006), Construction of Escherichia coli K-12 in-frame, single-gene knockout mutants: the Keio collection. Mol Syst Biol doi:10.1038/msb400050.), was carried out in 96-deep-well plates with 940 μl Minimal medium or 940 μl LB. Inoculation was performed by transferring cells from frozen stock cultures with a 96-well stamp (Kuhner, Birsfelden, Switzerland). Plates were incubated on an orbital shaker (300 rpm, 5 cm amplitude) at 25° C. for 48 h. Typically an OD₆₂₀ₙₘ of 2-4 was reached.

### Preparation of Cell Lysates

### Preparation of Lysis Buffer

The lysis buffer contained the following ingredients:

TABLE 7

| | | |
|---|---|---|
| 1M MOPS pH 7.5 | 5 | ml |
| DNAse I grade II (Roche) | 10 | mg |
| Lysozyme | 200 | mg |
| MgSO₄•7H₂O | 123.2 | mg |
| dithiothreitol (DTT) | 154.2 | mg |
| H₂O (MilliQ) | Balance to 100 | ml |

The solution was freshly prepared directly before use.

### Preparation of Cell Free Extract by Lysis

Cells from small scales growth (see previous paragraph) were harvested by centrifugation and the supernatant was discarded. The cell pellets formed during centrifugation were frozen at −20° C. for at least 16 h and then thawed on ice. 500 μl of freshly prepared lysis buffer were added to each well and cells were resuspended by vigorously vortexing the plate for 2-5 min. To achieve lysis, the plate was incubated at room temperature for 30 min. To remove cell debris, the plate was centrifuged at 4° C. and 6000 g for 20 min. The supernatant was transferred to a fresh plate and kept on ice until further use.

Enzymatic reactions for conversion of alpha-ketopimelic acid to alpha-aminopimelic acid In vitro

A reaction mixture was prepared comprising 50 mM alpha-ketopimelic acid, 100 mM alfa-methylbenzylamine and 0.1 mM pyridoxal 5′-phosphate in 50 mM potassium phosphate buffer, pH 7.5. 510 μl of the reaction mixture were dispensed into each well of the well plates. To start the reaction, 490 μl of the cell lysates were added, to each of the wells. Reaction mixtures were incubated on a shaker at 28° C. for 24 h. Furthermore, a chemical blank mixture (without cell free extract) and a biological blank (E. coli TOP10 with pBAD/Myc-His C) were incubated under the same conditions. Samples were analysed by HPLC-MS as described previously. The results are summarised in the following tables.

TABLE 8
production in vitro using lysates from cells grown in minimal medium.

| Deleted gene in | | | | | | | |
|---|---|---|---|---|---|---|---|
| the E. coli mutant strain | | | | KEIO plate | | | |
| Protein | alternative | E.C. | | position | | | mM |
| ID | name | Number | Accession | Plate | Row | Col | AAP |
| BioA | | 2.6.1.62 | NP_415295 | 44 | H | 2 | 0.01 |
| SerC | | 2.6.1.52 | NP_415427 | 41 | C | 4 | 0.01 |
| AspC | | 2.6.1.1 | NP_415448 | 41 | E | 4 | 0.01 |
| MalY | | 4.4.1.8 | NP_416139 | 52 | D | 2 | 0.01 |
| HisC | | 2.6.1.9 | NP_416525 | 41 | F | 7 | 0.01 |
| GlyA | | 2.1.2.1 | NP_417046 | 41 | B | 9 | 0.01 |
| Kbl | | 2.3.1.29 | NP_418074 | 47 | F | 10 | 0.01 |
| MetB | | 2.5.1.48 | NP_418374 | 41 | A | 12 | 0.01 |
| IlvE | | 2.6.1.42 | NP_418218 | 42 | E | 11 | 0.01 |
| YfhO | iscS | 2.8.1.7 | NP_417025 | 65 | D | 6 | 0.01 |
| Blanc | | | | | | | <0.01 |

| | |
|---|---|
| Wild-type | 0.23 |
| BW25113 | |

References to gene and protein can be found via www.ncbi.nlm.nih.gov/ (as available on 3 Sep. 2010)

TABLE 9
AAP production in vitro using lysates from cells grown in LB medium.

| Deleted gene in | | | | | | | |
|---|---|---|---|---|---|---|---|
| the E. coli mutant strain | | | | KEIO plate | | | |
| Protein | alternative | E.C. | | position | | | mM |
| ID | name | Number | Accession | Plate | Row | Col | AAP |
| B1680 | | 4.4.1.16 | NP_416195 | 63 | E | 3 | 0.28 |
| GoaG | | 2.6.1.19 | NP_415818 | 41 | D | 5 | 0.42 |
| SerC | | 2.6.1.52 | NP_415427 | 42 | C | 4 | 0.16 |
| SerC | | 2.6.1.52 | NP_415427 | 41 | C | 4 | 0.47 |
| AspC | | 2.6.1.1 | NP_415448 | 41 | E | 4 | 0.46 |
| YgjG | | 2.6.1.13 | NP_417544 | 69 | A | 9 | 0.55 |
| YbjU | | 4.1.2.5 | NP_415391 | 41 | B | 4 | 0.56 |
| TnaA | | 4.1.99.1 | NP_418164 | 64 | E | 9 | 0.43 |
| CstC | | 2.6.1. | NP_416262 | 83 | C | 8 | 0.6 |
| MetB | | 2.5.1.48 | NP_418374 | 42 | A | 12 | 0.55 |
| IlvE | | 2.6.1.42 | NP_418218 | 41 | E | 11 | 0.44 |
| YfhO | iscS | 2.8.1.7 | NP_417025 | 65 | D | 6 | 0.12 |
| Blanc | | | | | | | 0.03 |

| | |
|---|---|
| Wild-type | 3 |
| BW25113 | |

References to gene and protein can be found via www.ncbi.nlm.nih.gov/ (as available on 3 Sep. 2010)

From the results presented in Table 8 and in Table 9 it is clear that enzymes, present in the wild-type BW25113 lysate, are able to convert AKP to AAP. This activity is severely reduced in extracts, prepared from single-gene knockout mutants.

### Example 7

### Conversion of AKP to AAP in E. coli

Growth of E. coli

E. coli strains, identified as the E. coli KEIO mutant library, were grown overnight in LB were grown overnight in tubes with 10 ml 2XTY medium. 200 μl culture was transferred to shake flasks with 20 ml 2XTY medium. Flasks were incubated in an orbital shaker at 30° C. and 280 rpm. After 4 h cells from 20 ml culture were collected by centrifugation, resuspended in 4 ml 2XTY medium with 500 mg/l AKP and incubated in a 24 wells plate for 24 h at 30° C. and 210 rpm. After 24 hours the supernatant was collected by centrifugation and stored at −20 C for analysis.

### UPLC-MS/MS Analysis Method for the Determination of AAP

A Waters HSS T3 column 1.8 μm, 100 mm×2.1 mm was used for the separation of 6-ACA and AAP with gradient elution as depicted in Table 10. Eluent A consists of LC/MS grade water, containing 0.1% formic acid, and eluent B consists of acetonitrile, containing 0.1% formic acid. The flow-rate was 0.25 ml/min and the column temperature was kept constant at 40° C.

TABLE 10
gradient elution program used for the separation of AAP

Time (min)

| | 0 | 5.0 | 5.5 | 10 | 10.5 | 15 |
|---|---|---|---|---|---|---|
| % A | 100 | 85 | 20 | 20 | 100 | 100 |
| % B | 0 | 15 | 80 | 80 | 0 | 0 |

A Waters micromass Quattro micro API was used in electrospray either positive or negative ionization mode, depending on the compounds to be analyzed, using multiple reaction monitoring (MRM). The ion source temperature was kept at 130° C., whereas the desolvation temperature is 350° C., at a flow-rate of 500 L/hr.

For AAP the protonated molecule was fragmented with 13 eV, resulting in specific fragments from losses of H₂O, NH₃ and CO.

To determine concentrations, a calibration curve of external standards of synthetically prepared compounds was run to calculate a response factor for the respective ions. This was used to calculate the concentrations in samples. Samples were diluted appropriately (2-10 fold) in eluent A to overcome ion suppression and matrix effects.

### Analysis of Supernatant

Supernatant were diluted 5 times with water prior to UPLC-MS/MS analysis. Results (table 11) shows that in the wild-type E. coli strain BW25113 130 mg/l AAP is produced during the 24 hours incubation. E. coli strains with single gene deletions clearly show a reduced AAP production indicating that the genes deleted in these strains are directly or indirectly involved in the conversion of AKP into AAP.

TABLE 11
Conversion of AKP into AAP using E. coli mutant strains.

| Deleted gene in | | | | | | | |
|---|---|---|---|---|---|---|---|
| the E. coli mutant strain | | | | KEIO plate | | | AAP |
| Protein | alternative | E.C. | | position | | | (mg/ |
| ID | name | Number | Accession | Plate | Row | Col | l) |
| HisC | | 2.6.1.9 | NP_416525 | 42 | F | 7 | 32 |
| Kbl | | 2.3.1.29 | NP_418074 | 47 | F | 10 | 3 |
| Kbl | | 2.3.1.29 | NP_418074 | 48 | F | 10 | 9 |
| B1439 | ydcR | | NP_415956 | 23 | H | 9 | 35 |
| SerC | | 2.6.1.52 | NP_415427 | 41 | C | 4 | 22 |
| YhfS | | | NP_417835 | 36 | H | 5 | 25 |
| YgjG | | 2.6.1.13 | NP_417544 | 69 | A | 9 | 6 |
| MalY | | 4.4.1.8 | NP_416139 | 51 | D | 2 | 8 |
| MalY | | 4.4.1.8 | NP_416139 | 52 | D | 2 | 7 |
| CstC | | 2.6.1. | NP_416262 | 83 | C | 8 | 7 |
| AvtA | | 2.6.1.66 | NP_418029 | 42 | G | 10 | 17 |
| IlvE | | 2.6.1.42 | NP_418218 | 42 | E | 11 | 29 |
| YfhO | iscS | 2.8.1.7 | NP_417025 | 65 | D | 6 | 8 |
| BioF | | 2.3.1.47 | NP_415297 | 44 | B | 3 | 37 |
| SelA | | 2.9.1.1 | NP_418048 | 62 | H | 5 | 3 |
| YgjG | | 2.6.1.13 | NP_417544 | 70 | A | 9 | 14 |
| B2253 | yfbE | | NP_416756 | 77 | B | 10 | 16 |
| GabT | | 2.6.1.19 | NP_417148 | 83 | A | 11 | 27 |
| blanc | | | | | | | nd |

| | |
|---|---|
| Wild-type | 130 |
| BW25113 | |

Thus, a biocatalyst comprising one or more of these proteins referred to in Table 8, 9 or 11, or homologues thereof having AKP aminotransferase activity may advantageously be used in a method according to the invention. Such protein may be over-expressed, based on technology known in the art or described herein above.

1. Method for preparing 6-aminocaproic acid, comprising decarboxylating alpha-aminopimelic acid, using at least one biocatalyst comprising an enzyme having alpha-aminopimelic acid decarboxylase activity, wherein said enzyme comprises an amino acid sequence selected from the group of sequences represented by any of the SEQUENCE ID NO's: 2, 5, 8 and 11 and homologues of said sequences having alpha-aminopimelic acid decarboxylase activity.

2. Method according to claim 1, wherein said enzyme comprises a homologue having at least 40%, preferably at least 60%, in particular at least 80%, more in particular at least 90% sequence identity with any of the SEQUENCE ID NO's: 2, 5, 8 and 11.

3. Method according to claim 1, comprising preparing alpha-aminopimelic acid from alpha-ketopimelic acid.

4. Method according to claim 3, wherein the preparation of alpha-aminopimelic acid is catalysed by a biocatalyst in the presence of an amino donor, said biocatalyst having catalytic activity with respect to the transamination or the reductive amination of alpha-ketopimelic acid.

5. Method according to claim 4, wherein the biocatalyst comprises an enzyme having catalytic activity with respect to the transamination or the reductive amination of alpha-ketopimelic acid selected from the group of aminotransferases (E.C. 2.6.1) and amino acid dehydrogenases (E.C. 1.4.1).

6. Method according to claim 5, wherein the aminotransferase or amino acid dehydrogenase is selected from the group of β-aminoisobutyrate:α-ketoglutarate aminotransferases, β-alanine aminotransferases, aspartate aminotransferases, 4-amino-butyrate aminotransferases (EC 2.6.1.19), L-lysine 6-aminotransferase (EC 2.6.1.36), 2-aminoadipate aminotransferases (EC 2.6.1.39), 5-aminovalerate aminotransferases (EC 2.6.1.48), 2-aminohexanoate aminotransferases (EC 2.6.1.67), lysine:pyruvate 6-aminotransferases (EC 2.6.1.71), and lysine-6-dehydrogenases (EC 1.4.1.18).

7. Method according to claim 3, wherein an aminotransferase is used comprising an amino acid sequence according to Sequence ID NO 15, Sequence ID NO 18, Sequence ID NO 21, Sequence ID NO 23, Sequence ID NO 26, Sequence ID NO 28, Sequence ID NO 30, Sequence ID NO 32, Sequence ID NO 34, Sequence ID NO 36, Sequence ID NO 38, Sequence ID NO 40 Sequence ID NO 42, Sequence ID NO 44, Sequence ID NO 46, an aminotransferase mentioned in Table 8, 9 or 11 of the description, or a homologue of any of these sequences.

8. Method for preparing caprolactam, comprising cyclising the 6-aminocaproic acid prepared by a method according to claim 1, thereby forming caprolactam.

9. A recombinant host cell comprising a gene encoding a heterologous enzyme having alpha-aminopimelic acid decarboxylase activity, wherein said enzyme comprises an amino acid sequence represented by any of the SEQUENCE ID NO's: 2, 5, 8 and 11 and homologues of said sequences.

10. A recombinant host cell according to claim 9, comprising a nucleic acid sequence encoding the enzyme having alpha-aminopimelic acid decarboxylase activity, the nucleic acid sequence comprising a sequence according to any of the SEQUENCE ID NO's: 1, 3, 4, 6, 7, 9 and 10 and functional analogues thereof.

11. A recombinant host cell according to claim 9, comprising a nucleic acid sequence encoding a biocatalyst capable of catalysing a transamination reaction or a reductive amination reaction whereby alpha-aminopimelic acid is formed from alpha-ketopimelic acid.

12. A recombinant host cell according to claim 11, wherein the nucleic acid sequence encoding a biocatalyst capable of catalysing a transamination reaction or a reductive amination reaction is selected from the group of Sequence ID NO 15, Sequence ID NO 18, Sequence ID NO 21, Sequence ID NO 23, Sequence ID NO 26, Sequence ID NO 28, Sequence ID NO 30, Sequence ID NO 32, Sequence ID NO 34, Sequence ID NO 36, Sequence ID NO 38, Sequence ID NO 40 Sequence ID NO 42, Sequence ID NO 44, Sequence ID NO 46 or a homologue of any of these sequences.

13. Polynucleotide comprising a sequence according to any of the SEQUENCE ID NO's: 3, 6, and 9 and functional analogues thereof having a similar, the same or a better level of expression in an Escherichia host cell.
